(19) United States
(12) Patent Application Publication
(10) Pub. No.: US 2018/0327396 A1
(43) Pub. Date: Nov. 15, 2018

(54) NOVEL PYRIDINE DERIVATIVES

(71) Applicant: Hoffmann-La Roche Inc., Little Falls, NJ (US)

(72) Inventors: Luca Gobbi, Buus (CH); Uwe Grether, Efringen-Kirchen (DE); Matthias Nettekoven, Grenzach-Wyhlen (DE); Stephan Roever, Inzlingen (DE); Mark Rogers-Evans, Bottmingen (CH)

(73) Assignee: Hoffmann-La Roche Inc., Little Falls, NJ (US)

(21) Appl. No.: 16/044,320

(22) Filed: Jul. 24, 2018

Related U.S. Application Data
(63) Continuation of application No. 15/260,826, filed on Sep. 9, 2016.
Application No. 15/260,826 is a continuation of application No. PCT/EP2015/057151, filed on Apr. 1, 2015.

(30) Foreign Application Priority Data
Apr. 4, 2014 (EP) EP14163555.7

(51) Int. Cl. C07D 413/12; C07D 417/06; C07D 413/14; C07D 401/04; C07D 401/12

(52) U.S. Cl. CPC C07D 413/12; C07D 417/06; C07D 401/12; C07D 401/04; C07D 413/14

(57) ABSTRACT

The invention relates to a compound of formula (I)

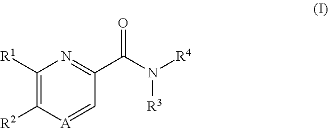

wherein A and R¹ to R⁴ are defined as in the description and in the claims. The compound of formula (I) can be used as a medicament.

### CROSS REFERENCE TO RELATED APPLICATIONS

This application is a continuation of International Application No. PCT/EP2015/057151 having an International Filing Date of 1 Apr. 2015, the entire contents of which are incorporated herein by reference, and which claims the benefit of priority under 35 U.S.C. § 119 to EP 14163555.7, filed 4 Apr. 2014.

### FIELD OF THE INVENTION

The present invention relates to organic compounds useful for therapy and/or prophylaxis in a mammal, and in particular to compounds that are preferential inverse agonists of the Cannabinoid Receptor 2.

### SUMMARY OF THE INVENTION

The present invention relates in particular to a compound of formula (I)

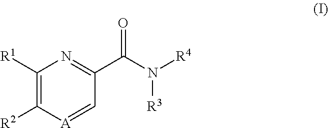

wherein

A is —CH— or nitrogen;

R^{l} is halophenyl, halophenylalkyl, haloalkoxy, halogen, alkoxyalkoxy, oxopyrrolidinyl or cycloalkylalkoxy;

R² is hydrogen, halophenylamino, cycloalkyl or haloazetidinyl;

one of R³ and R⁴ is hydrogen and the other one is —(CR⁵R⁶)ₘ—(CH₂)ₙ—R⁷;

or R³ and R⁴ together with the nitrogen atom to which they are attached form aminocarbonylthiomorpholinyl;

R⁵ and R⁶ are independently selected from hydrogen and alkyl;

R⁷ is 5-cycloalkyl-1,3,4-oxadiazolyl, 3-cycloalkyl-1,2,4-oxadiazolyl, 5-phenyl-1,3,4-oxadiazolyl, 3-phenyl-1,2,4-oxadiyzolyl, 5-alkyl-1,3,4-oxadiazolyl, 3-alkoxyalkoxyalkyl-1,2-oxazolyl, 1-hydroxyalkylpyrazolyl, 3-hydroxy-1-adamantyl, alkoxycarbonylmorpholinyl, 3-oxanyloxyalkyl-1,2-oxazol-5-yl, 3-azidoalkyl-1,2-oxazol-5-yl or 5-(4-fluorophenyl)-1,3,4-oxadiazolyl;

m is 0 or 1;

n is 0 or 1;

or a pharmaceutically acceptable salt or ester thereof;

provided that 6-chloro-N-(5-cyclopropyl-1,3,4-oxadiazol-2-yl)-2-pyridinecarboxamide is excluded.

The compound of formula (I) is particularly useful in the treatment or prophylaxis of pain, neuropathic pain, asthma, osteoporosis, inflammation, psychiatric diseases, psychosis, oncology, encephalitis, malaria, allergy, immunological disorders, arthritis, gastrointestinal disorders, psychiatric disorders rheumatoid arthritis, psychosis and allergy.

The cannabinoid receptors are a class of cell membrane receptors belonging to the G protein-coupled receptor superfamily. There are currently two known subtypes, termed Cannabinoid Receptor 1 (CB1) and Cannabinoid Receptor 2 (CB2). The CB1 receptor is mainly expressed in the central nervous (i.e. amygdala cerebellum, hippocampus) system and to a lesser amount in the periphery. CB2, which is encoded by the CNR2 gene, is mostly expressed peripherally, on cells of the immune system, such as macrophages and T-cells (Ashton, J. C. et al. Curr Neuropharmacol 2007, 5 (2), 73-80; Miller, A. M. et al. Br J Pharmacol 2008, 153 (2), 299-308; Centonze, D., et al. Curr Pharm Des 2008, 14 (23), 2370-42), and in the gastrointestinal system (Wright, K. L. et al. Br J Pharmacol 2008, 153 (2), 263-70). The CB2 receptor is also widely distributed in the brain where it is found primarily on microglia and not neurons (Cabral, G. A. et al. Br J Pharmacol 2008, 153 (2): 240-51).

The interest in CB2 receptor ligands has been steadily on the rise during the last decade (currently 30-40 patent applications/year). Evidences from different sources support the view that lipid endocannabinoid signaling through CB2 receptors represents an aspect of the mammalian protective armamentarium (Pacher, P. Prog Lipid Res 2011, 50, 193). Its modulation by either selective CB2 receptor agonists or inverse agonists/antagonists (depending on the disease and its stage) holds unique therapeutic potential in a huge number of diseases. For CB2 inverse agonists/antagonists therapeutic opportunities have been demonstrated for many pathological conditions including pain (Pasquini, S. J Med Chem 2012, 55 (11): 5391), neuropathic pain (Garcia-Gutierrez, M. S. Br J Pharmacol 2012, 165 (4): 951), psychiatric disorders (Garcia-Gutierrez, M. S. Br J Pharmacol 2012, 165 (4): 951), psychosis (Garcia-Gutierrez, M. S. Br J Pharmacol 2012, 165 (4): 951), osteoporosis and inflammation (Sophocleous, A. Calcif Tissue Int 2008, 82 (Suppl. 1):Abst OC18), psychiatric diseases and psychosis (Garcia-Gutierrez, M. S. Br J Pharmacol 2012, 165 (4): 951), oncology (Preet, A. Cancer Prey Res 2011, 4: 65), encephalitis and malaria (Zimmer, A. WO 2011045068), allergy and inflammation (Ueda, Y. Life Sci 2007, 80 (5): 414), encephalitis and malaria (Zimmer, WO 2011045068), asthma (Lunn, C. A. J Pharmacol Exp Ther 2006, 316 (2): 780), immunological disorders (Fakhfouri, G. Neuropharmacology 2012, 63 (4): 653), rheumatoid arthritis (Chackalamannil, S. U.S. Pat. No. 7,776,889), arthritis (Lunn, C. A. J Pharmacol Exp Ther 2006, 316 (2): 780), and gastrointestinal disorders (Barth, F. FR 2887550),

The compounds of the invention bind to and modulate the CB2 receptor and have lower CB1 receptor activity.

### DEFINITIONS

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C1-C8 alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl, isopropyl, butyl, isobutyl, tert.-butyl and pentyl. Methyl is a particular example of alkyl in the compound of formula (I).

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl and cyclohexyl, cycloheptyl and cyclooctyl. Particular examples of “cycloalkyl” are cyclopropyl, cyclobutyl and cyclopentyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec-butoxy and tert.-butoxy. Particular “alkoxy” are methoxy, ethoxy and tert.-butoxy.

The term “oxy”, alone or in combination, signifies the —O— group.

The term “oxo”, alone or in combination, signifies the ═O group.

The terms “halogen” or “halo”, alone or in combination, signifies fluorine, chlorine, bromine or iodine and particularly fluorine, chlorine or bromine, more particularly fluorine and chlorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. A particular “haloalkyl” is trifluoroethyl.

The term “haloalkoxy” or “haloalkyloxy”, alone or in combination, denotes an alkoxy group substituted with at least one halogen, particularly substituted with one to five halogens, particularly one to three halogens. A particular “haloalkoxy” is trifluoroethoxy.

The terms “hydroxyl” and “hydroxy”, alone or in combination, signify the —OH group.

The term “carbonyl”, alone or in combination, signifies the —C(O)— group.

The term “amino”, alone or in combination, signifies the primary amino group (—NH2), the secondary amino group (—NH—), or the tertiary amino group (—N—).

The term “aminocarbonyl”, alone or in combination, signifies the —C(O)—NH2, —C(O)—NH— or —C(O)—N— group.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

“Pharmaceutically acceptable esters” means that the compound of general formula (I) may be derivatised at functional groups to provide derivatives which are capable of conversion back to the parent compounds in vivo. Examples of such compounds include physiologically acceptable and metabolically labile ester derivatives, such as methoxymethyl esters, methylthiomethyl esters and pivaloyloxymethyl esters. Additionally, any physiologically acceptable equivalents of the compound of general formula (I), similar to the metabolically labile esters, which are capable of producing the parent compound of general formula (I) in vivo, are within the scope of this invention.

If one of the starting materials or compounds of formula (I) contain one or more functional groups which are not stable or are reactive under the reaction conditions of one or more reaction steps, appropriate protecting groups (as described e.g. in “Protective Groups in Organic Chemistry” by T. W. Greene and P. G. M. Wuts, 3rd Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compound of formula (I) can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

The term “asymmetric carbon atom” means a carbon atom with four different substituents. According to the Cahn-Ingold-Prelog Convention an asymmetric carbon atom can be of the “R” or “S” configuration.

### DETAILED DESCRIPTION OF EXEMPLARY EMBODIMENTS

The invention relates in particular to:

A compound or formula (I) wherein R¹ is halogen or cycloalkylalkoxy;

A compound or formula (I) wherein R¹ is chloro or cyclopropylmethoxy;

A compound or formula (I) wherein R² is hydrogen, halophenylamino or cycloalkyl;

A compound or formula (I) wherein R² is hydrogen, dichlorophenylamino or cyclopropyl;

A compound or formula (I) wherein R² is halophenylamino or cycloalkyl;

A compound or formula (I) wherein R² is dichlorophenylamino or cyclopropyl;

A compound or formula (I) wherein R² is hydrogen;

A compound or formula (I) wherein R⁵ and R⁶ are both alkyl at the same time;

A compound or formula (I) wherein R⁵ and R⁶ are both methyl at the same time; and

A compound or formula (I) wherein R⁷ is 5-phenyl-1,3,4-oxadiyzolyl, 3-alkoxyalkoxyalkyl-1,2-oxazolyl or 3-azidoalkyl-1,2-oxazol-5-yl.

The invention further relates to a compound or formula (I) selected from:

6-(4-chlorophenyl)-N-[1-(5-cyclopropyl-1,3,4-oxadiazol-2-yl)-2-methylpropan-2-yl]pyridine-2-carboxamide;

6-(4-chlorophenyl)-N-[2-methyl-1-(5-phenyl-1,3,4-oxadiazol-2-yl)propan-2-yl]pyridine-2-carboxamide;

N-[2-methyl-1-(5-phenyl-1,3,4-oxadiazol-2-yl)propan-2-yl]-6-(2,2,2-trifluoroethoxy)pyridine-2-carboxamide;

6-(4-chlorophenyl)-N-[2-methyl-1-(3-phenyl-1,2,4-oxadiazol-5-yl)propan-2-yl]pyridine-2-carboxamide;

N-[2-methyl-1-(3-phenyl-1,2,4-oxadiazol-5-yl)propan-2-yl]-6-(2,2,2-trifluoroethoxy)pyridine-2-carboxamide;

6-(3-chlorophenyl)-N-[2-methyl-1-(5-phenyl-1,3,4-oxadiazol-2-yl)propan-2-yl]pyridine-2-carboxamide;

6-(3-chlorophenyl)-N-[1-(3-cyclopropyl-1,2,4-oxadiazol-5-yl)-2-methylpropan-2-yl]pyridine-2-carboxamide;

6-(3-chlorophenyl)-N-[2-methyl-1-(3-phenyl-1,2,4-oxadiazol-5-yl)propan-2-yl]pyridine-2-carboxamide;

6-chloro-5-(2,4-dichloroanilino)-N-[2-methyl-1-(5-phenyl-1,3,4-oxadiazol-2-yl)propan-2-yl]pyridine-2-carboxamide;

6-(4-chlorophenyl)-5-cyclopropyl-N-[2-methyl-1-(5-phenyl-1,3,4-oxadiazol-2-yl)propan-2-yl]pyridine-2-carboxamide;

6-(2-methoxyethoxy)-N-[2-methyl-1-(3-phenyl-1,2,4-oxadiazol-5-yl)propan-2-yl]pyridine-2-carboxamide;

N-[2-methyl-1-(3-phenyl-1,2,4-oxadiazol-5-yl)propan-2-yl]-6-(2-oxopyrrolidin-1-yl)pyridine-2-carboxamide;

6-(3-chlorophenyl)-N-[1-(5-cyclobutyl-1,3,4-oxadiazol-2-yl)-2-methylpropan-2-yl]pyridine-2-carboxamide;

6-(2,4-dichlorophenyl)-N-[2-(5-phenyl-1,3,4-oxadiazol-2-yl)propan-2-yl]pyridine-2-carboxamide;

6-(2,4-dichlorophenyl)-N-[2-(5-methyl-1,3,4-oxadiazol-2-yl)propan-2-yl]pyridine-2-carboxamide;

6-(cyclopropylmethoxy)-5-(3,3-difluoroazetidin-1-yl)-N-[3-[1-(2-methoxyethoxy)-2-methylpropan-2-yl]-1,2-oxazol-5-yl]pyridine-2-carboxamide;

5-cyclopropyl-6-(cyclopropylmethoxy)-N-[1-(1-hydroxy-2-methylpropan-2-yl)pyrazol-4-yl]pyridine-2-carboxamide;

5-cyclopropyl-6-(cyclopropylmethoxy)-N-[3-[1-(2-methoxyethoxy)-2-methylpropan-2-yl]-1,2-oxazol-5-yl]pyridine-2-carboxamide;

5-cyclopropyl-6-(cyclopropylmethoxy)-N-[3-(1-hydroxy-2-methylpropan-2-yl)-1,2-oxazol-5-yl]pyridine-2-carboxamide;

5-cyclopropyl-6-(cyclopropylmethoxy)-N-[3-[1-(2-ethoxyethoxy)-2-methylpropan-2-yl]-1,2-oxazol-5-yl]pyridine-2-carboxamide;

6-(cyclopropylmethoxy)-5-(3,3-difluoroazetidin-1-yl)-N-(3-hydroxy-1-adamantyl)pyridine-2-carboxamide;

tert-butyl 2-[[[5-cyclopropyl-6-[(4-fluorophenyl)methyl]pyridine-2-carbonyl]amino]methyl]morpholine-4-carboxylate;

(3 S)-4-[5-cyclopropyl-6-(cyclopropylmethoxy)pyrazine-2-carbonyl]thiomorpholine-3-carboxamide;

5-cyclopropyl-6-(cyclopropylmethoxy)-N-[3-(1-hydroxy-2-methylpropan-2-yl)-1,2-oxazol-5-yl]pyrazine-2-carboxamide;

5-cyclopropyl-6-(cyclopropylmethoxy)-N-[3-[2-methyl-1-(oxan-2-yloxy)propan-2-yl]-1,2-oxazol-5-yl]pyrazine-2-carboxamide;

N-[3-(1-azido-2-methylpropan-2-yl)-1,2-oxazol-5-yl]-5-cyclopropyl-6-(cyclopropylmethoxy)pyrazine-2-carboxamide;

6-(Cyclopropylmethoxy)-5-(3,3-difluoroazetidin-1-yl)-N-[1-[5-(4-fluorophenyl)-1,3,4-oxadiazol-2-yl]-2-methylpropan-2-yl]pyridine-2-carboxamide;

5-Cyclopentyl-6-(cyclopropylmethoxy)-N-[1-[5-(4-fluorophenyl)-1,3,4-oxadiazol-2-yl]-2-methylpropan-2-yl]pyridine-2-carboxamide;

5-Cyclopentyl-6-(cyclopropylmethoxy)-N-[3-[1-(2-methoxyethoxy)-2-methylpropan-2-yl]-1,2-oxazol-5-yl]pyridine-2-carboxamide; and

5-Cyclopentyl-6-(cyclopropylmethoxy)-N-[3-[1-(2-ethoxyethoxy)-2-methylpropan-2-yl]-1,2-oxazol-5-yl]pyridine-2-carboxamide.

The invention particularly relates to a compound or formula (I) selected from:

6-chloro-5-(2,4-dichloroanilino)-N-[2-methyl-1-(5-phenyl-1,3,4-oxadiazol-2-yl)propan-2-yl]pyridine-2-carboxamide;

5-cyclopropyl-6-(cyclopropylmethoxy)-N-[3-[1-(2-methoxyethoxy)-2-methylpropan-2-yl]-1,2-oxazol-5-yl]pyridine-2-carboxamide;

5-cyclopropyl-6-(cyclopropylmethoxy)-N-[3-[1-(2-ethoxyethoxy)-2-methylpropan-2-yl]-1,2-oxazol-5-yl]pyridine-2-carboxamide; and

N-[3-(1-azido-2-methylpropan-2-yl)-1,2-oxazol-5-yl]-5-cyclopropyl-6-(cyclopropylmethoxy)pyrazine-2-carboxamide.

The synthesis of the compound of formula (I) can, for example, be accomplished according to the following schemes.

Unless indicated otherwise, A and R¹-R⁴ have in the following schemes the meaning as defined above.

Following the procedure according to scheme 1, compound AA (X═Cl, Br, I or trifluoromethanesulfonate; R′═H, methyl, ethyl, isopropyl, tert. butyl or another suitable protecting group described for example in T. W. Greene et al., Protective Groups in Organic Chemistry, John Wiley and Sons Inc. New York 1999, 3^{rd} edition,) can be used as starting material. AA is either commercially available, described in the literature, can be synthesized by a person skilled in the art or as described in the experimental part.

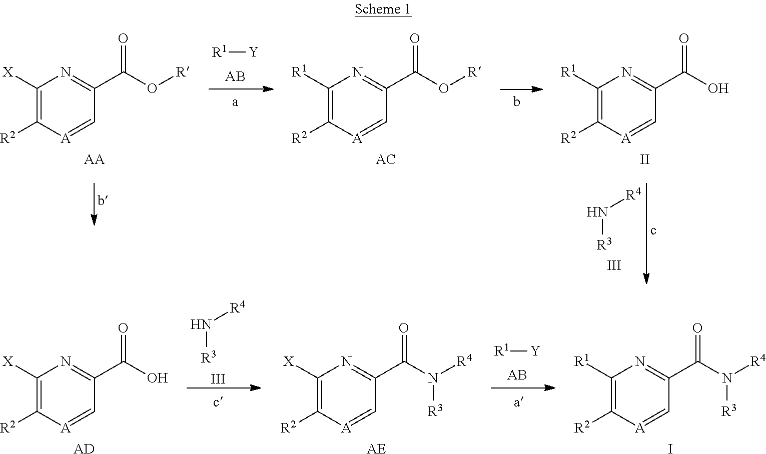

Compound AC can be prepared from AA by coupling a suitably substituted aryl or arylalkyl metal species of formula AB (Y is e.g. a trifluoroborate group like [BF₃]⁻K⁺, a boronic acid group B(OH)₂ or a boronic acid pinacol ester group) (step a), particularly an arylboronic acid or arylboronic acid ester in the presence of a suitable catalyst, in particular a palladium catalyst and more particularly palladium(II)acetate/triphenylphosphine mixtures or palladium(III)chloride-dppf (1,1′-bis(diphenylphosphino)ferrocene) complexes and a base such as triethylamine, sodium carbonate or potassium phosphate in an inert solvent such as dimethylformamide, toluene, tetrahydrofuran, acetonitrile or dimethoxyethane.

Alternatively compound AC can be prepared from AA by coupling an oxopyrrolidinyl species of formula AB (Y is H) (step a), in the presence of a suitable catalyst, in particular a palladium catalyst and more particularly tris(dibenzylideneacetone)dipalladium(0) complexes and a base such as triethylamine, sodium carbonate or cesium carbonate in an inert solvent such as dimethylformamide, toluene, tetrahydrofuran, acetonitrile or dimethoxyethane.

Alternatively compound AA can be transformed into compound AC by reaction with a suitably substituted primary or secondary alcohol AB (Y is H) in the presence of a base, for example sodium hydride or potassium hydroxide, with or without an inert solvent, for example DMF or DMSO, at temperatures ranging from room temperature to the reflux temperature of the solvent, particularly at room temperature.

The saponification of the ester of general formula AC (R′≠H) by methods well known to the ones skilled in the art—using e.g. aqueous LiOH, NaOH or KOH in tetrahydrofuran/ethanol or another suitable solvent at temperatures between 0° C. and the reflux temperature of the solvent employed—leads to an acid of general formula II (step b).

Compound I can be prepared from II and the corresponding amine of formula III by suitable amide bond forming reactions (step c). These reactions are known in the art. For example coupling reagents like N,N′-carbonyl-diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU), and O-benzotriazole-N,N,N′,N′-tetramethyl-uronium-hexafluoro-phosphate (HBTU) can be employed to affect such transformation. A convenient method is to use for example HBTU and a base, for example N-methylmorpholine in an inert solvent such as for example dimethylformamide at room temperature.

Alternatively esters of general formula AA (R′≠H) can be saponified by methods well known to the ones skilled in the art—using e.g. aqueous LiOH, NaOH or KOH in tetrahydrofuran/ethanol or another suitable solvent at temperatures between 0° C. and the reflux temperature of the solvent employed—to give acids of general formula AD (step b′).

Compounds AE can be prepared from AD and the corresponding amine of formula III by suitable amide bond forming reactions (step c′). These reactions are known in the art. For example coupling reagents like N,N′-carbonyl-diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU) or O-benzotriazole-N,N,N′,N′-tetramethyl-uronium-hexafluoro-phosphate (HBTU) can be employed to affect such transformation. A convenient method is to use for example HBTU and a base, for example N-methylmorpholine in an inert solvent such as for example dimethylformamide at room temperature.

Compound I can be prepared from AE by coupling a suitably substituted aryl or arylalkyl metal species of formula AB (Y is e.g. a trifluoroborate group like [BF₃]⁻K⁺, a boronic acid group B(OH)₂ or a boronic acid pinacol ester group) (step a′), particularly an arylboronic acid or arylboronic acid ester in the presence of a suitable catalyst, in particular a palladium catalyst and more particularly palladium(II)acetate/triphenylphosphine mixtures or palladium(II)chloride-dppf (1,1′-bis(diphenylphosphino)ferrocene) complexes and a base such as triethylamine, sodium carbonate or potassium phosphate in an inert solvent such as dimethylformamide, toluene, tetrahydrofuran, acetonitrile or dimethoxyethane.

Alternatively compound I can be prepared from AE by coupling an oxopyrrolidinyl species of formula AB (Y is H) (step a′), in the presence of a suitable catalyst, in particular a palladium catalyst and more particularly tris(dibenzylideneacetone)dipalladium(0) complexes and a base such as triethylamine, sodium carbonate or cesium carbonate in an inert solvent such as dimethylformamide, toluene, tetrahydrofuran, acetonitrile or dimethoxyethane.

Alternatively compound AE can be transformed to compounds I by reaction with a suitably substituted primary or secondary alcohol AB (Y is H) in the presence of a base, for example sodium hydride or potassium hydroxide, with or without an inert solvent, for example DMF or DMSO, at temperatures ranging from room temperature to the reflux temperature of the solvent, particularly at room temperature.

Amines III are either commercially available, described in the literature, can be synthesized by a person skilled in the art or as described in the experimental part.

If one of the starting materials, compounds of formulae AA, AB or III, contains one or more functional groups which are not stable or are reactive under the reaction conditions of one or more reaction steps, appropriate protecting groups (P) (as described e.g. in T. W. Greene et al., Protective Groups in Organic Chemistry, John Wiley and Sons Inc. New York 1999, 3^{rd} edition) can be introduced before the critical step applying methods well known in the art. Such protecting groups can be removed at a later stage of the synthesis using standard methods known in the art.

If one or more compounds of formulae AA to AE, II or III contain chiral centers, compounds of formula I can be obtained as mixtures of diastereomers or enantiomers, which can be separated by methods well known in the art, e.g. (chiral) HPLC or crystallization.

Racemic compounds can e.g. be separated into their antipodes via diastereomeric salts by crystallization or by separation of the antipodes by specific chromatographic methods using either a chiral adsorbent or a chiral eluent.

Following the procedure according to scheme 2, compound BA (3,5-dibromo-2-pyrazinamine, CAN 24241-18-7) can be used as starting material for the synthesis of compounds I-a wherein A is nitrogen and R^{1′} is halophenyl, halophenylalkyl or oxopyrrolidinyl.

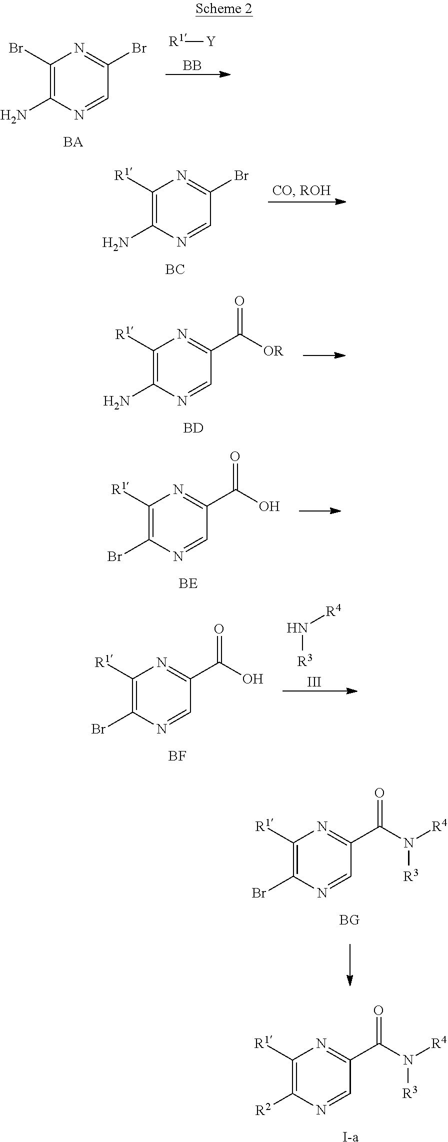

Compound BC can be prepared from BA by coupling a suitably substituted aryl or arylalkyl metal species of formula BB (Y is e.g. a trifluoroborate group like [BF₃]⁻K⁺, a boronic acid B(OH)₂ or a boronic acid pinacol ester group), particularly an arylboronic acid or arylboronic acid ester in the presence of a suitable catalyst, in particular a palladium catalyst and more particularly tetrakis(triphenylphosphine)-palladium(0) and a base such as triethylamine or potassium phosphate, in particular sodium carbonate, in an inert solvent such as dimethylformamide, toluene, tetrahydrofurane, acetonitrile or in particular dimethoxyethane, at temperatures from room temperature to the boiling point of the solvent mixture.

Alternatively compound BC can be prepared from BA by coupling an oxopyrrolidinyl species of formula BB (Y is H), in the presence of a suitable catalyst, in particular a palladium catalyst or more particularly tris(dibenzylideneacetone)dipalladium(0) complexes, and a base such as triethylamine, sodium carbonate or cesium carbonate, in an inert solvent such as dimethylformamide, toluene, tetrahydrofuran, acetonitrile or dimethoxyethane.

Compounds of the general formula BD can be obtained from compounds of the general formula BC by palladium (II), particularly palladium(II) acetate catalyzed carbonylation in the presence of a suitable base such as a tertiary amine base, particularly triethylamine, in a suitable solvent such as an alcohol, particularly methanol.

Compounds of the general formula BE can be obtained from compounds of the general formula BD by reaction with nitrosating agents such as a metal nitrite or an organic nitrite more particularly isoamylnitrite, in the presence of a bromide source such as hydrobromic acid or more particularly trimethylbromosilane in a suitable solvent such as halogenated hydrocarbons more particularly dibromomethane.

The saponification of the ester of general formula BE by methods well known to the ones skilled in the art—using e.g. aqueous LiOH, NaOH or KOH in tetrahydrofuran/ethanol or another suitable solvent at temperatures between 0° C. and the reflux temperature of the solvent employed—leads to an acid of general formula BF.

Compound BG can be prepared from BF and the corresponding amine of formula III by suitable amide bond forming reactions. These reactions are known in the art. For example coupling reagents like N,N′-carbonyl-diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU) or O-benzotriazole-N,N,N′,N′-tetramethyl-uronium-hexafluoro-phosphate (HBTU) can be employed to affect such transformation. Alternative methods known in the art may commence by preparing the acid chloride from BF and coupling with an amine of formula III in the presence of a suitable base. A convenient method is to use for example 1-chloro-N,N,2-trimethylpropenylamine and a base, for example N-ethyl-N-isopropylpropan-2-amine (DIEA), in an inert solvent such as for example dimethylformamide at room temperature.

Amines III are either commercially available, described in the literature, can be synthesized by a person skilled in the art or obtained as described in the experimental part.

Compounds I-a wherein R² is cycloalkyl can be prepared from BG by coupling a suitably substituted cycloalkyl or cycloalkenyl metal species, particularly a cyclopropyl metal species, like cyclopropylzinc(II) chloride, or cyclopropylboronic acid or cyclopropyltrifluoro-borate salts with BG in the presence of a suitable catalyst, particularly a palladium catalyst like tetrakis-(triphenyl-phosphine)palladium, or [1,3-bis(2,6-diisopropylphenyl)imidazol-2-ylidene](3-chloropyridyl)-palladium(II) dichloride, or palladium(II)acetate, in an inert solvent such as THF or toluene at room temperature up to the reflux temperature of the solvent. The person skilled in the art will appreciate that for coupling the cycloalkyl- or cycloalkenyl-boron species the addition of a suitable base, like potassium phosphate, is necessary for the reaction to commence. In cases where the practitioner skilled in the art chooses to couple with a cycloalkenyl metal species, like cycloalkenylboronic acid esters, compounds I-a will be obtained only after an additional hydrogenation step, for example by hydrogenation with hydrogen gas in the presence of a palladium catalyst, for example palladium on charcoal, in an inert solvent, for example ethanol, at suitable temperatures and pressures, particularly at ambient temperature and pressure.

Compounds I-a wherein R² is haloazetidinyl can be prepared from BG by reacting with the corresponding azetidine in the presence of a base, particularly DBU or triethylamine, in an inert solvent, particularly DMSO or dioxane at temperatures ranging from room temperature to 45° C.

If one of the starting materials, compounds of formula III, contains one or more functional groups which are not stable or are reactive under the reaction conditions of one or more reaction steps, appropriate protecting groups (P) (as described e.g. in T. W. Greene et al., Protective Groups in Organic Chemistry, John Wiley and Sons Inc. New York 1999, 3^{rd} edition) can be introduced before the critical step applying methods well known in the art. Such protecting groups can be removed at a later stage of the synthesis using standard methods known in the art.

If one or more compounds of formula III contain chiral centers, pyridines of formula I-a can be obtained as mixtures of diastereomers or enantiomers, which can be separated by methods well known in the art, e.g. (chiral) HPLC or crystallization. Racemic compounds can e.g. be separated into their antipodes via diastereomeric salts by crystallization or by separation of the antipodes by specific chromatographic methods using either a chiral adsorbent or a chiral eluent.

Following the procedure according to scheme 3, compound BA (3,5-dibromo-2-pyrazinamine, CAN 24241-18-7) can be used as starting material for the synthesis of compounds I where R¹ is cycloalkylalkoxy, haloalkoxy or alkoxyalkoxy.

Compound BA can be transformed to compounds CB by reaction with a suitably substituted primary or secondary alcohol AB (Y is H) in the presence of a base, for example sodium hydride, with or without an inert solvent, for example DMF, at temperatures ranging from room temperature to the reflux temperature of the solvent, particularly at room temperature.

The Boc-protection of compounds of general formula CB by methods well known to the ones skilled in the art—using e.g. di-tert-butyl dicarbonate in an inert solvent, particularly dichloromethane in the presence of a catalytic amount of base, particularly dimethylaminopyridine—leads to compounds of general formula CC if an excess of di-tert-butyl dicarbonate is employed in the reaction.

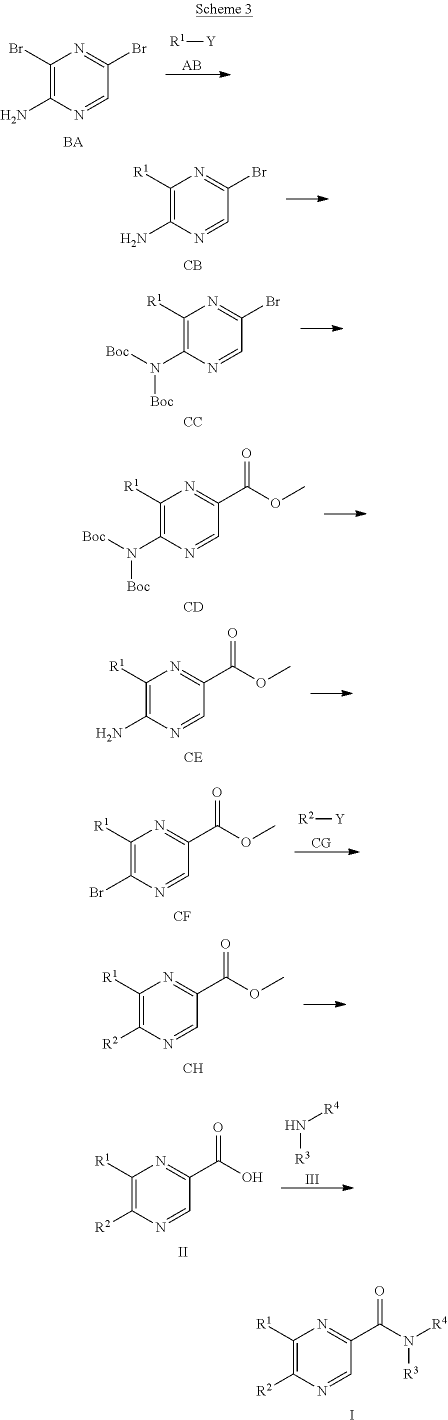

Compounds of the general formula CD can be obtained from compounds of the general formula CC by palladium (II), particularly palladium(II) acetate catalyzed carbonylation in the presence of a suitable base such as a tertiary amine base, particularly triethylamine, in a suitable solvent such as an alcohol, particularly methanol.

The solvolysis of boc-protected compounds of general formula CD by methods well known to the ones skilled in the art—using e.g. a protic solvent, particularly methanol at elevated temperatures, particularly reflux temperature—leads to compounds of general formula CE.

Compounds of the general formula CF can be obtained from compounds of the general formula CE by reaction with nitrosating agents such as a metal nitrite or an organic nitrite more particularly tert-butyl nitrite, in the presence of a bromide source such as hydrobromic acid or more particularly trimethylbromosilane in a suitable solvent such as halogenated hydrocarbons more particularly dibromomethane.

Compounds CH wherein R² is cycloalkyl can be prepared from CF by coupling a suitably substituted cycloalkyl or cycloalkenyl metal species CG (Y is e.g. a trifluoroborate group like [BF₃]⁻K⁺, a boronic acid group B(OH)₂ or a boronic acid pinacol ester group) particularly a cyclopropylboronic acid or cyclopropyltrifluoro-borate salt with CF in the presence of a suitable catalyst, particularly a palladium catalyst like palladium(II) acetate in the presence of cyclohexylphosphine in an inert solvent such as toluene at room temperature up to the reflux temperature of the solvent in the presence of a suitable base, like potassium phosphate. In cases where the practitioner skilled in the art chooses to couple with a cycloakenyl metal species, like cycloalkenylboronic acid esters, compounds CH will be obtained only after an additional hydrogenation step, for example by hydrogenation with hydrogen gas in the presence of a palladium catalyst, for example palladium on charcoal, in an inert solvent, for example ethanol, at suitable temperatures and pressures, particularly at ambient temperature and pressure.

Compounds CH where R² is haloazetidinyl can be prepared from CF by reacting with the corresponding haloazetidine CG (Y is H) in the presence of a base, particularly DBU or triethylamine, in an inert solvent, particularly DMSO or dioxane at temperatures ranging from room temperature to 45° C.

The saponification of the ester of general formula CII by methods well known to the ones skilled in the art—using e.g. aqueous LiOH, NaOH or KOH in tetrahydrofuran/ethanol or another suitable solvent at temperatures between 0° C. and the reflux temperature of the solvent employed—leads to the acid of general formula II.

Compounds of formula II can be further elaborated to compound I by coupling a compound of formula II-c with an amine of the formula III by amide coupling methods known in the art, as for example with the help of an amide coupling agent under basic conditions. For example coupling reagents like N,N′-carbonyl-diimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU) or O-benzotriazole-N,N,N′,N′-tetramethyl-uronium-hexafluoro-phosphate (HBTU) can be employed to affect such transformation. A convenient method is to use for example O-benzotriazole-N,N,N′,N′-tetramethyl-uronium-hexafluoro-phosphate (HBTU) and a base, for example N-ethyl-N-isopropylpropan-2-amine (DIEA) in an inert solvent such as for example dimethylformamide at room temperature. Alternative methods known in the art may commence by preparing the acid chloride from II and coupling with an amine of formula III in the presence of a suitable base.

Amines III are either commercially available, described in the literature, can be synthesized by a person skilled in the art or obtained as described in the experimental part.

If one of the starting materials, compounds of formulae BA, AB, CG or III, contains one or more functional groups which are not stable or are reactive under the reaction conditions of one or more reaction steps, appropriate protecting groups (P) (as described e.g. in T. W. Greene et al., Protective Groups in Organic Chemistry, John Wiley and Sons Inc. New York 1999, 3^{rd} edition) can be introduced before the critical step applying methods well known in the art. Such protecting groups can be removed at a later stage of the synthesis using standard methods known in the art.

If one or more compounds of formulae BA, AB, CG or III contain chiral centers, pyridines of formula I can be obtained as mixtures of diastereomers or enantiomers, which can be separated by methods well known in the art, e.g. (chiral) HPLC or crystallization. Racemic compounds can e.g. be separated into their antipodes via diastereomeric salts by crystallization or by separation of the antipodes by specific chromatographic methods using either a chiral adsorbent or a chiral eluent.

Following the procedure according to scheme 4, compound DA (R′═H, methyl, ethyl, isopropyl, tert. butyl or another suitable protecting group described for example in T. W. Greene et al., Protective Groups in Organic Chemistry, John Wiley and Sons Inc. New York 1999, 3^{rd} edition) can be used as starting material. DA is either commercially available (e.g. for R′=methyl: 5-bromo-6-chloro-pyridine-2-carboxylic acid methyl ester CAN 1214353-79-3), described in the literature or can be synthesized by a person skilled in the art.

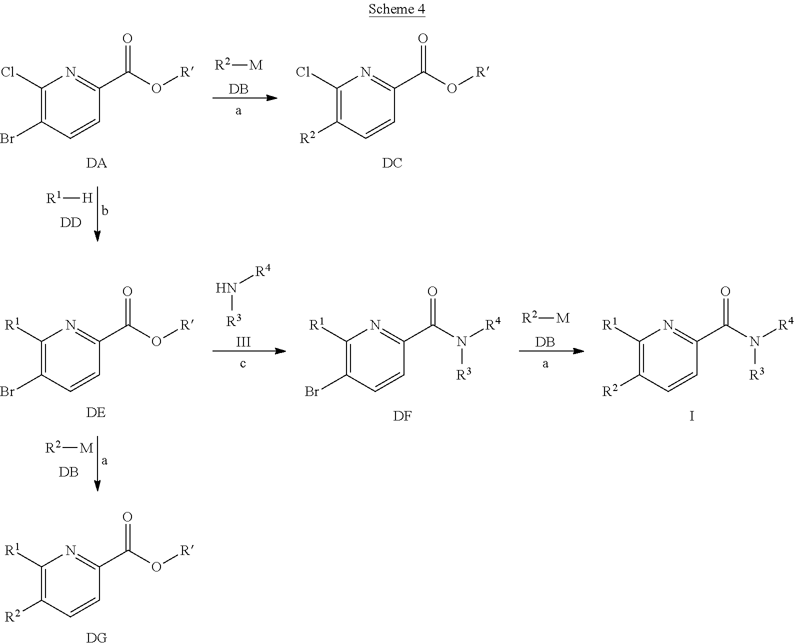

Compound DC can be prepared from DA by coupling a suitably substituted aryl, heteroaryl or alkenyl metal species of formula DB (M is e.g. a trifluoroborate group like [BF₃]⁻K⁺, a boronic acid group B(OH)₂ or a boronic acid pinacol ester group) (step a), e.g. an organotrifluoroborate potassium salt in the presence of a palladium catalyst such as palladium(II)acetate/butyl-1-adamantylphosphine and a base such as cesium carbonate in an inert solvent such as toluene at temperatures between 50° C. and the boiling temperature of the solvent, or an arylboronic acid or arylboronic acid ester in the presence of a suitable catalyst, in particular a palladium catalyst and more particularly palladium(II)acetate/triphenylphosphine mixtures or palladium(II)chloride-dppf (1,1′-bis(diphenylphosphino)ferrocene) complexes and a base such as triethylamine, sodium carbonate or potassium phosphate in an inert solvent such as dimethylformamide, toluene, tetrahydrofuran, acetonitrile or dimethoxyethane. Optionally, compound DB (M is H) can also be an amine or amide which is coupled to DA by methods well known to a person skilled in the art, e.g. using a palladium catalyst such as tris(dibenzylideneacetone)dipalladium/dimethylbisdiphenyl-phosphinoxanthene and a base such as cesium carbonate in a solvent such as 1,4-dioxane, preferentially at the boiling point of the solvent. Alternatively, compound DB can also be a sulfonamide (M is H) which undergoes a copper(I) mediated reaction with DA to form DC following procedures described in the literature, e.g. using copper(I) iodide and 1,3-di(pyridin-2-yl)propane-1,3-dione in the presence of a base such as potassium carbonate in a solvent such as dimethylformamide at elevated temperatures preferentially at the boiling point of the solvent. Optionally, alkenyl containing R² residues can be transformed to the corresponding alkyl congeners DC using conditions described in the literature such as e.g. a hydrogenation reaction using hydrogen gas in the presence of a catalyst such as palladium on carbon in a solvent such as ethanol or ethyl acetate particularly at ambient temperature.

Compound DC can be further elaborated to compound I by: i) reaction with compound DD to form compound DG as described in steps a and a′ of scheme 1; ii) saponification as described in step b of scheme 1; and iii) amide bond formation as described in step c of scheme 1.

Furthermore, compound DA can be converted into compound DE by treatment with compound DD as described in steps a and a′ of scheme 1 (step b).

Subsequent transformation of compound DE into compound DG can be achieved as discussed for the conversion of DA into DC (step a).

Compound DG can be further elaborated to compound I by: i) saponification as described in step b of scheme 1; ii) amide bond formation as described in step c of scheme 1.

Alternatively, compound DE (R′=methyl, ethyl, isopropyl, tert. butyl or another suitable protecting group described for example in T. W. Greene et al., Protective Groups in Organic Chemistry, John Wiley and Sons Inc. New York 1999, 3^{rd} edition) can be: i) converted into its acid congener DE (R′═H) as described in step b of scheme 1; ii) transformed into the corresponding amide DF by treatment with amine III as described in step c of scheme 1; and iii) reacted with DB as described in step a to arrive at compound I.

Furthermore, compound I can also be synthesized applying the following reaction sequence: i) saponification of compound DA (R′=methyl, ethyl, isopropyl, tert. butyl or another suitable protecting group described for example in T. W. Greene et al., Protective Groups in Organic Chemistry, John Wiley and Sons Inc. New York 1999, 3^{rd} edition) to its acid congener DE (R′═H) as described in step b of scheme 1; ii) conversion to the corresponding amide by treatment with amine III as described in step c of scheme 1; iii) reaction with compound DB as described in step a; and iv) reaction with compound DD as described in step c. Optionally step iii) and step iv) can be interchanged.

If one of the starting materials, compounds of formulae DA, DB or DD contains one or more functional groups which are not stable or are reactive under the reaction conditions of one or more reaction steps, appropriate protecting groups (P) (as described e.g. in T. W. Greene et al., Protective Groups in Organic Chemistry, John Wiley and Sons Inc. New York 1999, 3^{rd} edition) can be introduced before the critical step applying methods well known in the art. Such protecting groups can be removed at a later stage of the synthesis using standard methods known in the art.

If one or more compounds of formulae DA, DB or DD contain chiral centers, picolines of formula DC and DG can be obtained as mixtures of diastereomers or enantiomers, which can be separated by methods well known in the art, e.g. (chiral) HPLC or crystallization. Racemic compounds can e.g. be separated into their antipodes via diastereomeric salts by crystallization or by separation of the antipodes by specific chromatographic methods using either a chiral adsorbent or a chiral eluent.

Following the procedure according to scheme 5, commercially available 5-bromo-6-methyl-pyridine-2-carbonitrile EA (CAN 1173897-86-3) can be used as starting material. In scheme 5, R¹ is benzyl or halobenzyl; R^{1′} is phenyl or halophenyl.

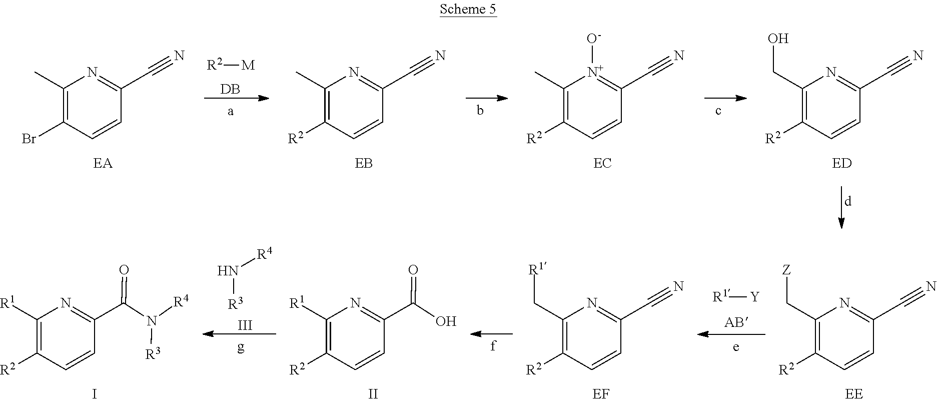

Compound EB can be prepared from EA by coupling a suitably substituted aryl, heteroaryl or alkenyl metal species of formula DB (Y is e.g. a trifluoroborate group like [BF₃]⁻K⁺, a boronic acid group B(OH)₂ or a boronic acid pinacol ester group) (step a), e.g. an organotrifluoroborate potassium salt in the presence of a palladium catalyst such as palladium(II)acetate/butyl-1-adamantylphosphine and a base such as cesium carbonate in an inert solvent such as toluene at temperatures between 50° C. and the boiling temperature of the solvent, or an arylboronic acid or arylboronic acid ester in the presence of a suitable catalyst, in particular a palladium catalyst, more particularly palladium(II)acetate/triphenylphosphine mixtures or palladium(II)chloride-dppf (1,1′-bis(diphenylphosphino)ferrocene) complexes and a base such as triethylamine, sodium carbonate or potassium phosphate in an inert solvent such as dimethylformamide, toluene, tetrahydrofuran, acetonitrile or dimethoxyethane. Optionally, compound DB can also be an amine or amide (Y is H) which is coupled to EA by methods well known to a person skilled in the art, e.g. using a palladium catalyst such as tris(dibenzylideneacetone)dipalladium/dimethylbisdiphenyl-phosphinoxanthene and a base such as cesium carbonate in a solvent such as 1,4-dioxane preferentially at the boiling point of the solvent. Optionally, alkenyl containing R² residues can be transformed to the corresponding alkyl congeners EA using conditions described in the literature such as e.g. a hydrogenation reaction using hydrogen gas in the presence of a catalyst such as palladium on carbon in a solvent such as ethanol or ethyl acetate particularly at ambient temperature.

Further transformation of EB to EC can be achieved by oxidation with a suitable oxidizing reagent under conditions known to a person skilled in the art, e.g. by treatment with 3-chloro perbenzoic acid in dichloromethane at ambient temperature (step b).

Conversion of N-oxide EC to alcohol ED can be performed under conditions well known to a person skilled in the art, e.g. by reaction with trifluoroacetic acid anhydride in a solvent such as dichloromethane preferentially at ambient temperature and subsequent treatment with a base such as sodium hydroxide (step c).

Reactions how to convert alcohol ED into compound EE containing a leaving group (Z═Cl, Br or another suitable leaving group) are well described in the literature and known to those skilled in the art (step d). For example alcohol ED can be transformed to compound EE with Z═Br by reaction with carbon tetrabromide and triphenylphosphine in a solvent such as tetrahydrofuran at temperatures between 0° C. and the boiling point of the solvent, preferentially at 40° C.

Conversion of compound EE to compound EF can e.g. be accomplished by coupling a suitably substituted aryl metal species of formula AB′ (Y is e.g. a boronic acid group B(OH)₂ or a boronic acid pinacol ester group), particularly an arylboronic acid or arylboronic acid ester in the presence of a suitable catalyst, in particular a palladium catalyst and more particularly palladium(II)acetate/triphenylphosphine mixtures or palladium(II)chloride-dppf (1,1′-bis(diphenylphosphino)ferrocene) complexes and a base such as triethylamine, cesium carbonate or potassium phosphate in an inert solvent such as dimethylformamide, toluene, tetrahydrofuran or 1,4-dioxane (step e).

Nitrile EF can be hydrolyzed to acid II (A=CH) under acidic or basic conditions known to a person skilled in the art, e.g. by treatment with an aqueous solution of sodium hydroxide at 100° C. (step f).

Further conversion of compound II to compound I can be done by applying amide bond formation conditions as depicted in step c of scheme 1 (step g).

If one of the starting materials, compounds of formulae EA, DB, AB′ or III, contains one or more functional groups which are not stable or are reactive under the reaction conditions of one or more reaction steps, appropriate protecting groups (P) (as described e.g. in T. W. Greene et al., Protective Groups in Organic Chemistry, John Wiley and Sons Inc. New York 1999, 3^{rd} edition) can be introduced before the critical step applying methods well known in the art. Such protecting groups can be removed at a later stage of the synthesis using standard methods known in the art.

If one or more compounds of formulae EA to EF, DB, AB′, II or III contain chiral centers, picolines of formula I can be obtained as mixtures of diastereomers or enantiomers, which can be separated by methods well known in the art, e.g. (chiral) HPLC or crystallization. Racemic compounds can e.g. be separated into their antipodes via diastereomeric salts by crystallization or by separation of the antipodes by specific chromatographic methods using either a chiral adsorbent or a chiral eluent.

The invention thus also relates to a process for the preparation of a compound of formula (I) comprising one of the following steps:

(a) the reaction of a compound of formula (A)

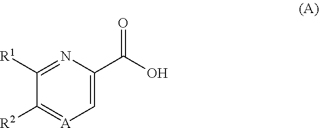

in the presence of NHR³R⁴, an amide coupling agent and a base, wherein A and R¹ to R⁴ are as defined above;

(b) the reaction of a compound of formula (B)

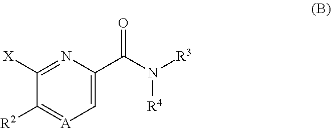

in the presence of R¹—Y, a palladium catalyst and a base, wherein X is Cl, Br, I or trifluoromethanesulfonate, Y is a trifluoroborate group, a boronic acid group or a boronic acid pinacol ester group, R¹ is halophenyl or halophenylalkyl and A and R² to R⁴ are as defined above; or

(c) the reaction of a compound of formula (C)

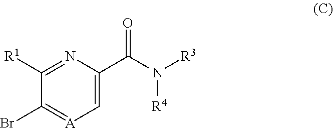

in the presence of R²-M, a palladium catalyst and a base, wherein R¹ is halophenyl, halophenylalkyl or oxopyrrolidinyl, R² is cycloalkyl, A and R³-R⁴ are as defined above and M is a trifluoroborate group, a boronic acid group or a boronic acid pinacol ester group.

In step (a), amide coupling agents for the reaction of compounds of formula (A) with amines of formula NHR³R⁴ are for example N,N′-carbonyldiimidazole (CDI), N,N′-dicyclohexylcarbodiimide (DCC), 1-(3-dimethylaminopropyl)-3-ethylcarbodiimide hydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT), O-benzotriazol-1-yl-N,N,N′,N′-tetramethyluronium tetrafluoroborate (TBTU), or O-benzotriazole-N,N,N′,N′-tetramethyl-uronium-hexafluoro-phosphate (HBTU). Particular coupling agents are TBTU and HATU.

In step (a), suitable bases include triethylamine, N-methylmorpholine and particularly diisopropylethylamine.

Alternative methods known in the art may commence by preparing the acid chloride from (A) and coupling with an amine of formula NHR³R⁴ in the presence of a suitable base.

In step (b), the palladium catalyst is for example palladium(II)acetate in the presence of cyclohexylphosphine.

In step (b), the base is for example potassium phosphate.

In step (c), the palladium catalyst is for example palladium(II)acetate in the presence of butyl-1-adamantylphosphine.

In step (c), the base is for example cesium carbonate.

The invention further relates to a compound of formula (I) when manufactured according to the above process.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The invention thus also relates to:

A compound of formula (I) for use as therapeutically active substance;

A pharmaceutical composition comprising a compound of formula (I) and a therapeutically inert carrier;

The use of a compound of formula (I) for the treatment or prophylaxis of pain, neuropathic pain, asthma, osteoporosis, inflammation, psychiatric diseases, psychosis, oncology, encephalitis, malaria, allergy, immunological disorders, arthritis, gastrointestinal disorders, psychiatric disorders rheumatoid arthritis, psychosis or allergy;

The use of a compound of formula (I) for the preparation of a medicament for the treatment or prophylaxis of pain, neuropathic pain, asthma, osteoporosis, inflammation, psychiatric diseases, psychosis, oncology, encephalitis, malaria, allergy, immunological disorders, arthritis, gastrointestinal disorders, psychiatric disorders rheumatoid arthritis, psychosis or allergy;

A compound of formula (I) for the treatment or prophylaxis of pain, neuropathic pain, asthma, osteoporosis, inflammation, psychiatric diseases, psychosis, oncology, encephalitis, malaria, allergy, immunological disorders, arthritis, gastrointestinal disorders, psychiatric disorders rheumatoid arthritis, psychosis or allergy; and

A method for the treatment or prophylaxis of pain, neuropathic pain, asthma, osteoporosis, inflammation, psychiatric diseases, psychosis, oncology, encephalitis, malaria, allergy, immunological disorders, arthritis, gastrointestinal disorders, psychiatric disorders rheumatoid arthritis, psychosis or allergy, which method comprises administering an effective amount of a compound of formula (I) to a patient in need thereof.

The invention will now be illustrated with the following examples which have no limiting character.

### EXAMPLES

Abbreviations

MS=mass spectrometry; EI=electron impact; ISP=ion spray, corresponds to ESI (electrospray); NMR data are reported in parts per million (δ) relative to internal tetramethylsilane and are referenced to the deuterium lock signal from the sample solvent (d₆-DMSO unless otherwise stated); coupling constants (J) are in Hertz, mp=melting point; bp=boiling point; DIEA=N-ethyl-N-isopropylpropan-2-amine; DMF=dimethylformamide; DMSO=dimethyl-sulfoxide; dppf=1,1′-bis(diphenylphosphino)ferrocene; EtOAc=ethyl acetate, HATU=2-(3H-[1,2,3]triazolo[4,5-b]pyridin-3-yl)-1,1,3,3-tetramethylisouronium hexafluorophosphate(V); HBTU=O-benzotriazole-N,N,N′,N′-tetramethyl-uronium-hexafluoro-phosphate; HPLC=LC=high performance liquid chromatography; iPrOAc=isopropyl acetate; m-CPBA=meta-chloroperoxybenzoic acid; Rt=retention time; TBTU=O-(benzotriazol-1-yl)-N,N,N′,N′-tetramethyl-uronium-tetrafluoroborate; TEMPO=2,2,6,6-tetra-methylpiperidine 1-oxyl radical; THF=tetrahydrofuran; tlc=thin layer chromatography.

### Example 1

### 6-(4-Chlorophenyl)-N-[1-(5-cyclopropyl-1,3,4-oxadiazol-2-yl)-2-methylpropan-2-yl]pyridine-2-carboxamide

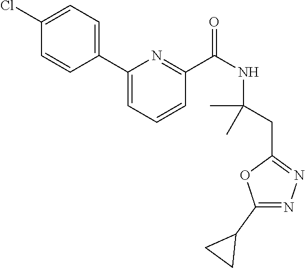 [SMILES: CC(C)(Cc1nnc(C2CC2)o1)NC(=O)c1cccc(-c2ccc(Cl)cc2)n1]

### a) tert-Butyl N-[4-[2-(cyclopropanecarbonyl)hydrazinyl]-2-methyl-4-oxobutan-2-yl]carbamate

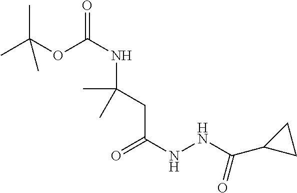 [SMILES: CC(C)(CC(=O)NNC(=O)C1CC1)NC(=O)OC(C)(C)C]

To a mixture of 3-[[(1,1-dimethylethoxy)carbonyl]amino]-3-methyl-butanoic acid (CAN 129765-95-3, 1.43 g, 6.59 mmol), DIEA (3.41 mL, 19.8 mmol) and TBTU (2.12 g, 6.59 mmol) in DMF (50 mL) was added cyclopropanecarboxylic acid hydrazide (CA 6952-93-8, 0.66 g, 6.59 mmol). The reaction mixture was stirred for 10 hours at room temperature and afterwards the solvent was removed in vacuo. The residue was dissolved in ethyl acetate (50 mL) and washed with saturated sodium bicarbonate solution (50 mL), 1 N hydrochloric acid (30 mL) and brine (30 mL). Water phases were extracted with ethyl acetate (50 mL), organic phases were pooled, dried with MgSO4, filtered and concentrated in vacuo to give the title compound (1.7 g, 77%) in approx. 90% purity as yellow oil; MS (ISP): 300.2 [MH⁺].

### b) tert-Butyl N-[1-(5-cyclopropyl-1,3,4-oxadiazol-2-yl)-2-methylpropan-2-yl]carbamate

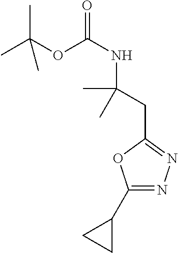 [SMILES: CC(C)(Cc1nnc(C2CC2)o1)NC(=O)OC(C)(C)C]

To a mixture of tert-butyl N-[4-[2-(cyclopropanecarbonyl)hydrazinyl]-2-methyl-4-oxobutan-2-yl]carbamate (1.70 g, 5.68 mmol) and triphenylphosphine (2.23 g, 8.52 mmol) in acetonitrile (60 mL) was added DIEA (2.98 mL, 17 mmol) and hexachloroethane (1.74 g, 7.38 mmol). The reaction mixture was stirred for 4 hours at room temperature and afterwards the solvent was removed in vacuo. The residue was dissolved in dichloromethane (80 mL) and washed with water (2×40 mL) and brine (40 mL). Water phases were extracted with dichloromethane (80 mL), organic phases were pooled, dried with MgSO4, filtered and concentrated in vacuo. The residue was purified by flash chromatography (silica, 0% to 100% ethyl acetate in heptane) to give the title compound (1.02 g, 64%) as white solid; MS (ISP): 282.2 [MH⁺].

### c) 1-(5-Cyclopropyl-1,3,4-oxadiazol-2-yl)-2-methylpropan-2-amine hydrochloride

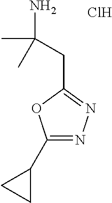

tert-Butyl N-[1-(5-cyclopropyl-1,3,4-oxadiazol-2yl)-2-methylpropan-2-yl]carbamate (1.02 g, 3.63 mmol) was dissolved in dioxane (14 mL) and a solution of 4 N HCl in dioxane (9.1 mL, 36.3 mmol) was added. The reaction mixture was stirred for 18 hours at room temperature and afterwards diluted with tert-butyl methylether (50 mL). The product precipitated and was isolated by filtration and subsequent drying in vacuo to give the title compound (718 mg, 91%) as white solid; MS (ISP): 182.1 [MH⁺].

### d) 6-(4-Chlorophenyl)-N-[1-(5-cyclopropyl-1,3,4-oxadiazol-2-yl)-2-methylpropan-2-yl]pyridine-2-carboxamide

A solution of 6-(4-chlorophenyl)-2-pyridinecarboxylic acid (CAN 135432-77-8, 0.2 mmol), 1-(5-cyclopropyl-1,3,4-oxadiazol-2-yl)-2-methylpropan-2-amine hydrochloride (0.2 mmol), DIEA (175 μL, 1 mmol) and TBTU (77.1 mg, 0.24 mmol) in DMF (0.5 mL) was stirred for 20 h at room temperature. The crude reaction mixture was concentrated in vacuo by centrifugation and purified by flash chromatography (silica gel, 0% to 100% ethyl acetate in heptane) to give the title compound (64 mg, 81%) as light-yellow solid; LC-MS (UV peak area/ESI) 99%, 397.1426 [MH⁺].

### Example 2

### 6-(4-Chlorophenyl)-N-[2-methyl-1-(5-phenyl-1,3,4-oxadiazol-2-yl)propan-2-yl]pyridine-2-carboxamide

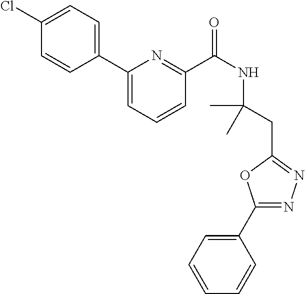 [SMILES: CC(C)(Cc1nnc(-c2ccccc2)o1)NC(=O)c1cccc(-c2ccc(Cl)cc2)n1]

### a) tert-Butyl N-[4-(2-benzoylhydrazinyl)-2-methyl-4-oxobutan-2-yl]carbamate

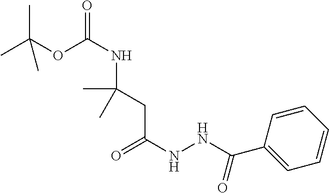 [SMILES: CC(C)(CC(=O)NNC(=O)c1ccccc1)NC(=O)OC(C)(C)C]

The title compound was synthesized in analogy to Example 1a, using 3-[[(1,1-dimethylethoxy)carbonyl]amino]-3-methyl-butanoic acid (CAN 129765-95-3, 1.36 g, 6.24 mmol) and benzoic acid hydrazide (CAN 613-94-5, 0.85 g, 6.24 mmol) as starting materials and isolated (1.97 g, 85%) in approx. 90% purity as orange oil, MS (ISP): 336.3 [MH⁺].

### b) tert-Butyl N-[2-methyl-1-(5-phenyl-1,3,4-oxadiazol-2-yl)propan-2-yl]carbamate

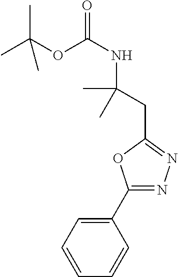 [SMILES: CC(C)(Cc1nnc(-c2ccccc2)o1)NC(=O)OC(C)(C)C]

The title compound was synthesized in analogy to Example 1b, using tert-butyl N-[4-(2-benzoylhydrazinyl)-2-methyl-4-oxobutan-2-yl]carbamate (Example 2a, 1.97 g, 5.87 mmol) as starting material and isolated (1.32 g, 71%) as white solid, MS (ISP): 318.1 [MH⁺].

### c) 2-Methyl-1-(5-phenyl-1,3,4-oxadiazol-2-yl)propan-2-amine hydrochloride

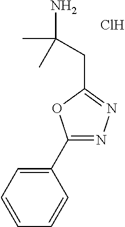 [SMILES: CC(C)(N)Cc1nnc(-c2ccccc2)o1 ; Cl]

The title compound was synthesized in analogy to Example 1c, using 6 tert-butyl N-[2-methyl-1-(5-phenyl-1,3,4-oxadiazol-2-yl)propan-2-yl]carbamate (Example 2b, 1.32 g, 4.16 mmol) as starting material and isolated (1.03 g, 98%) as white solid, MS (ISP): 218.1 [MH⁺].

### d) 6-(4-Chlorophenyl)-N-[2-methyl-1-(5-phenyl-1,3,4-oxadiazol-2-yl)propan-2-yl]pyridine-2-carboxamide

The title compound was synthesized in analogy to Example 1d, using 6-(4-chlorophenyl)-2-pyridinecarboxylic acid (CAN 135432-77-8, 0.2 mmol) and 2-methyl-1-(5-phenyl-1,3,4-oxadiazol-2-yl)propan-2-amine hydrochloride (Example 2c, 0.2 mmol) as starting materials and isolated (71 mg, 82%) as light yellow solid, LC-MS (UV peak area/ESI) 100%, 433.1417 [MH⁺].

### Example 3

### N-[2-Methyl-1-(5-phenyl-1,3,4-oxadiazol-2-yl)propan-2-yl]-6-(2,2,2-trifluoroethoxy)pyridine-2-carboxamide

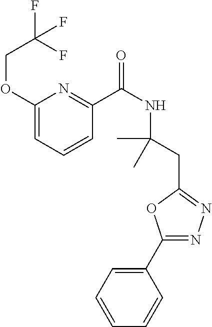 [SMILES: CC(C)(Cc1nnc(-c2ccccc2)o1)NC(=O)c1cccc(OCC(F)(F)F)n1]

### 6-(2,2,2-trifluoroethoxy)-2-pyridinecarboxylic acid (CAN 1247503-48-5)

The title compound was synthesized in analogy to Example 1d, using 6-(2,2,2-trifluoroethoxy)-2-pyridinecarboxylic acid (CAN 1247503-48-5, 0.2 mmol) and 2-methyl-1-(5-phenyl-1,3,4-oxadiazol-2-yl)propan-2-amine hydrochloride (Example 2c, 0.2 mmol) as starting materials and isolated (59 mg, 70%) as white solid, LC-MS (UV peak area/ESI) 99%, 421.1475 [MH⁺].

### Example 4

### 6-(4-Chlorophenyl)-N-[2-methyl-1-(3-phenyl-1,2,4-oxadiazol-5-yl)propan-2-yl]pyridine-2-carboxamide

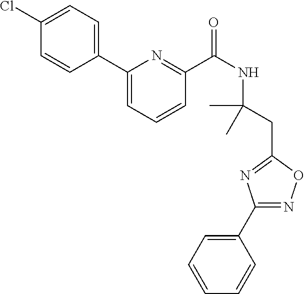 [SMILES: CC(C)(Cc1nc(-c2ccccc2)no1)NC(=O)c1cccc(-c2ccc(Cl)cc2)n1]

The title compound was synthesized in analogy to Example 1d, using 6-(4-chlorophenyl)-2-pyridinecarboxylic acid (CAN 135432-77-8, 0.2 mmol) and α,α-dimethyl-3-phenyl-1,2,4-oxadiazole-5-ethanamine hydrochloride (1:1) (CAN 1426444-03-2, 0.2 mmol) as starting materials and isolated (83 mg, 96%) as light yellow solid, LC-MS (UV peak area/ESI) 100%, 433.1421 [MH⁺].

### Example 5

### N-[2-Methyl-1-(3-phenyl-1,2,4-oxadiazol-5-yl)propan-2-yl]-6-(2,2,2-trifluoroethoxy)pyridine-2-carboxamide

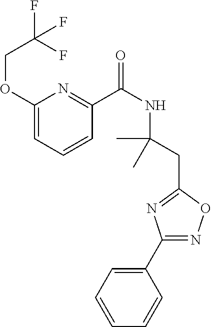 [SMILES: CC(C)(Cc1nc(-c2ccccc2)no1)NC(=O)c1cccc(OCC(F)(F)F)n1]

The title compound was synthesized in analogy to Example 1d, using 6-(2,2,2-trifluoroethoxy)-2-pyridinecarboxylic acid (CAN 1247503-48-5, 0.2 mmol) and α,α-dimethyl-3-phenyl-1,2,4-oxadiazole-5-ethanamine hydrochloride (1:1) (CAN 1426444-03-2, 0.2 mmol) as starting materials and isolated (66 mg, 79%) as white solid, LC-MS (UV peak area/ESI) 98%, 421.1476 [MH⁺].

### Example 6

### 6-(3-Chlorophenyl)-N-[2-methyl-1-(5-phenyl-1,3,4-oxadiazol-2-yl)propan-2-yl]pyridine-2-carboxamide

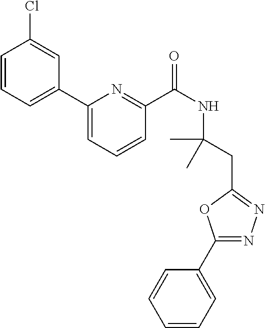 [SMILES: CC(C)(Cc1nnc(-c2ccccc2)o1)NC(=O)c1cccc(-c2cccc(Cl)c2)n1]

The title compound was synthesized in analogy to Example 1d, using 6-(3-chlorophenyl)-2-pyridinecarboxylic acid (CAN 863704-38-5, 0.2 mmol) and 2-methyl-1-(5-phenyl-1,3,4-oxadiazol-2-yl)propan-2-amine hydrochloride (Example 2c, 0.2 mmol) as starting materials and isolated (84 mg, 97%) as white solid, LC-MS (UV peak area/ESI) 95%, 433.1431 [MH⁺].

### Example 7

### 6-(3-Chlorophenyl)-N-[1-(3-cyclopropyl-1,2,4-oxadiazol-5-yl)-2-methylpropan-2-yl]pyridine-2-carboxamide

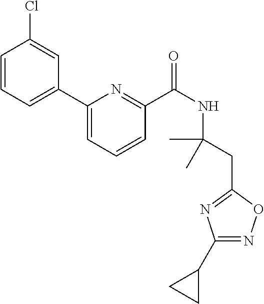 [SMILES: CC(C)(Cc1nc(C2CC2)no1)NC(=O)c1cccc(-c2cccc(Cl)c2)n1]

The title compound was synthesized in analogy to Example 1d, using 6-(3-chlorophenyl)-2-pyridinecarboxylic acid (CAN 863704-38-5, 0.2 mmol) and 3-cyclopropyl-α,α-dimethyl-1,2,4-oxadiazole-5-ethanamine (CAN1341734-01-7, 0.2 mmol) as starting materials and isolated (73 mg, 92%) as orange solid, LC-MS (UV peak area/ESI) 100%, 397.1434 [MH⁺].

### Example 8

### 6-(3-Chlorophenyl)-N-[2-methyl-1-(3-phenyl-1,2,4-oxadiazol-5-yl)propan-2-yl]pyridine-2-carboxamide

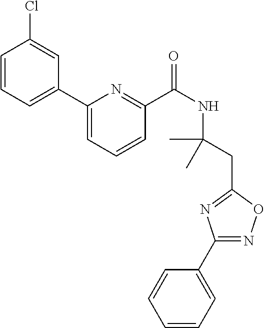 [SMILES: CC(C)(Cc1nc(-c2ccccc2)no1)NC(=O)c1cccc(-c2cccc(Cl)c2)n1]

The title compound was synthesized in analogy to Example 1d, using 6-(3-chlorophenyl)-2-pyridinecarboxylic acid (CAN 863704-38-5, 0.2 mmol) and α,α-dimethyl-3-phenyl-1,2,4-oxadiazole-5-ethanamine hydrochloride (1:1) (CAN 1426444-03-2, 0.2 mmol) as starting materials and isolated (83 mg, 96%) as white solid, LC-MS (UV peak area/ESI) 100%, 433.1428 [MH⁺].

### Example 9

### 6-Chloro-5-(2,4-dichloroanilino)-N-[2-methyl-1-(5-phenyl-1,3,4-oxadiazol-2-yl)propan-2-yl]pyridine-2-carboxamide

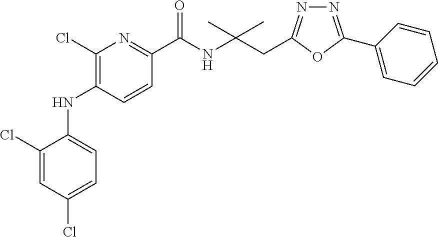 [SMILES: CC(C)(Cc1nnc(-c2ccccc2)o1)NC(=O)c1ccc(Nc2ccc(Cl)cc2Cl)c(Cl)n1]

### a) 6-Chloro-5-(2,4-dichloroanilino)pyridine-2-carboxylic acid methyl ester

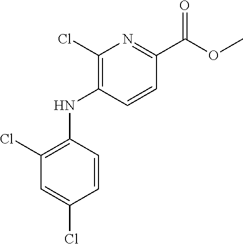 [SMILES: COC(=O)c1ccc(Nc2ccc(Cl)cc2Cl)c(Cl)n1]

Under an argon atmosphere a mixture of palladium(II)acetate (4.4 mg, 19 μmol) and 2-(dicyclohexylphosphino)biphenyl (13.6 mg, 39 μmol) in dioxane (1.9 mL) was stirred for 10 min at ambient temperature and added to a suspension of methyl 5,6-dichloropyridine-2-carboxylate (CAN 1214375-24-2, 100 mg, 485 μmol), 2,4-dichloroaniline (CAN 554-00-7, 78.6 mg, 485 μmol) and K₂CO₃ (1.34 g, 9.71 mmol) in dioxane (3.24 mL). The reaction mixture was heated to reflux and stirred for 20 h, poured into 20 mL ice/brine and extracted with iPrOAc (2×50 mL). The organic layers were washed with ice/brine (1×50 mL), dried over Na₂SO₄ and concentrated in vacuo to give 108 mg of a brown oil. The crude product was purified by preparative TLC (2 mm SiO₂ layer, with heptane/iPrOAc 9:1, elution with iPrOAc) to give the title compound (6 mg, 18 μmol, 4%) as brown solid.

### b) 6-Chloro-5-(2,4-dichloroanilino)pyridine-2-carboxylic acid

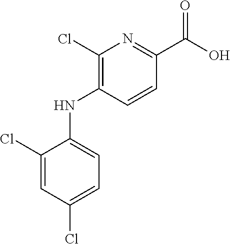 [SMILES: O=C(O)c1ccc(Nc2ccc(Cl)cc2Cl)c(Cl)n1]

Lithium hydroxide hydrate (911 μg, 22 μmol) was added to a solution of 6-chloro-5-(2,4-dichloroanilino)pyridine-2-carboxylic acid methyl ester (6 mg, 18 μmol) in THF (49 μL) and water (25 μL). The reaction mixture was stirred at ambient temperature for 20 h, poured onto 1 M HCl/icewater (20 mL) and extracted with iPrOAc (2×25 mL). The combined extracts were washed with ice/water (2×25 mL) and dried over Na₂SO₄. The solvent was removed under reduced pressure to give the title compound (6 mg, 19 μmol, quant.) as offwhite solid which was sufficiently pure to be used in the next reaction step, MS (ISP): 314.8 [MH⁻].

### c) 6-Chloro-5-(2,4-dichloroanilino)-N-[2-methyl-1-(5-phenyl-1,3,4-oxadiazol-2-yl)propan-2-yl]pyridine-2-carboxamide

The title compound was synthesized in analogy to Example 1d, using 6-chloro-5-(2,4-dichloroanilino)pyridine-2-carboxylic acid (19 μmol) and 2-methyl-1-(5-phenyl-1,3,4-oxadiazol-2-yl)propan-2-amine hydrochloride (Example 2c, 21 μmol) as starting materials and isolated (7 mg, 57%) as colorless oil, LC-MS: 518.0724 [MH⁺].

### Example 10

### 6-(4-Chlorophenyl)-5-cyclopropyl-N-[2-methyl-1-(5-phenyl-1,3,4-oxadiazol-2-yl)propan-2-yl]pyridine-2-carboxamide

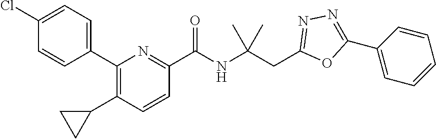 [SMILES: CC(C)(Cc1nnc(-c2ccccc2)o1)NC(=O)c1ccc(C2CC2)c(-c2ccc(Cl)cc2)n1]

### a) 6-(4-Chlorophenyl)-5-cyclopropyl-pyridine-2-carboxylic acid methyl ester

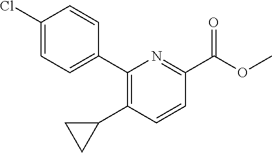 [SMILES: COC(=O)c1ccc(C2CC2)c(-c2ccc(Cl)cc2)n1]

A suspension of methyl methyl 6-chloro-5-cyclopropyl-pyridine-2-carboxylate (CAN 1415898-27-9, 100 mg, 472 μmol), 4-chlorophenylboronic acid (CAN 1679-18-1, 88.7 mg, 567 μmol), 2 M aqueous sodium carbonate solution (472 μL, 945 μmol) and 1,1′-bis(diphenylphosphino)ferrocene-palladium(II)dichloride dichloromethane complex (19.3 mg, 23.6 μmol) in toluene (1.5 mL) was heated to 90° C. for 30 h under an argon atmosphere. The reaction mixture was filtered through Speedex®. The solvent was removed under reduced pressure to give 145 mg of brown crystals which were purified by flash-chromatography (5 g SiO₂, heptan/0-30% iPrOAc in 75min) to give the title compound (92 mg, 68%) as off-white crystals, MS (ISP): 288.2 [MH⁺].

### b) 6-(4-Chlorophenyl)-5-cyclopropyl-pyridine-2-carboxylic acid

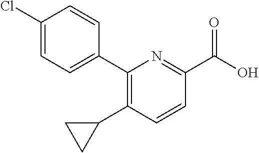 [SMILES: O=C(O)c1ccc(C2CC2)c(-c2ccc(Cl)cc2)n1]

In analogy to the procedure described in Example 9 b, 6-(4-chlorophenyl)-5-cyclopropyl-pyridine-2-carboxylic acid methyl ester (313 μmol) was hydrolyzed to give the title compound (102 mg, quant.) as colorless oil, MS (ISP): 272.1 [MH⁻].

### c) 6-(4-Chlorophenyl)-5-cyclopropyl-N-[2-methyl-1-(5-phenyl-1,3,4-oxadiazol-2-yl)propan-2-yl]pyridine-2-carboxamide

The title compound was synthesized in analogy to Example 1d, using 6-(4-chlorophenyl)-5-cyclopropyl-pyridine-2-carboxylic acid (37 μmol) and 2-methyl-1-(5-phenyl-1,3,4-oxadiazol-2-yl)propan-2-amine hydrochloride (Example 2c, 37 μmol) as starting materials and isolated (13 mg, 75%) as colorless oil, MS (ISP): 473.3 [MH⁺].

### Example 11

### 6-(2-Methoxyethoxy)-N-[2-methyl-1-(3-phenyl-1,2,4-oxadiazol-5-yl)propan-2-yl]pyridine-2-carboxamide

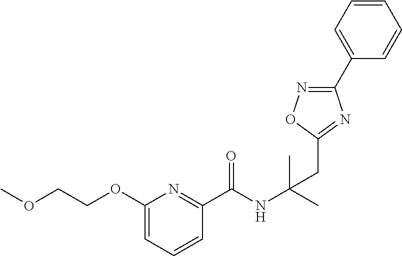 [SMILES: COCCOc1cccc(C(=O)NC(C)(C)Cc2nc(-c3ccccc3)no2)n1]

The title compound was synthesized in analogy to Example 1d, using 6-(2-methoxyethoxy)-2-pyridinecarboxylic acid (CAN 1248697-20-2, 0.1 mmol) and α,α-dimethyl-3-phenyl-1,2,4-oxadiazole-5-ethanamine hydrochloride (1:1) (CAN 1426444-03-2, 0.1 mmol) as starting materials and isolated (33 mg, 97%) as colorless oil, LC-MS (UV peak area/ESI) 100%, 397.1866 [MH⁺].

### Example 12

### N-[2-Methyl-1-(3-phenyl-1,2,4-oxadiazol-5-yl)propan-2-yl]-6-(2-oxopyrrolidin-1-yl)pyridine-2-carboxamide

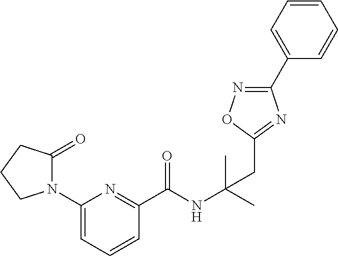 [SMILES: CC(C)(Cc1nc(-c2ccccc2)no1)NC(=O)c1cccc(N2CCCC2=O)n1]

### a) 6-(2-Oxopyrrolidin-1-yl)pyridine-2-carboxylic acid methylester

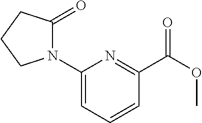 [SMILES: COC(=O)c1cccc(N2CCCC2=O)n1]

To a red suspension of 6-chloro-2-pyridinecarboxylic acid methyl ester (CAN 6636-55-1, 515 mg, 3 mmol), cesium carbonate (1.47 g, 4.5 mmol), 4,5-bis(diphenylphosphino)-9,9-dimethylxanthene (17.4 mg, 0.03 mmol) and tris(dibenzylideneacetone)dipalladium (0) (27.5 mg, 0.03 mmol) in dioxane (5 mL) was added 2-pyrrolidone (511 mg, 6 mmol). The reaction mixture was microwaved twice for 30 minutes at 140° C., cooled and partitioned between ethyl acetate and brine. Organic phases were pooled, dried with MgSO4, filtered and concentrated in vacuo. The residue was purified by flash chromatography (silica gel, 50% ethyl acetate in heptane) to give the title compound (860 mg, quant.) as white solid that was used without further purification in the next step; LC-MS (UV peak area/ESI) 94%, 221.0922 [MH⁺].

### b) 6-(2-Oxopyrrolidin-1-yl)pyridine-2-carboxylic acid

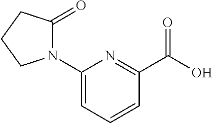 [SMILES: O=C(O)c1cccc(N2CCCC2=O)n1]

A solution of 6-(2-oxopyrrolidin-1-yl)pyridine-2-carboxylic acid methylester (911 mg, 4.11 mmol), and lithiumhydroxide (297 mg, 12.4 mmol) in THF (85 mL) and water (25 mL) was stirred at 0° C. for 3 hours. The reaction mixture was poured onto 1 N hydrochloric acid (200 mL) and extracted with ethyl acetate (2×200 mL). Organic phases were pooled, dried with MgSO4, filtered and concentrated in vacuo. The residue was purified by flash chromatography (silica gel, ethyl acetate) to give the title compound (212 mg, 25%) as light yellow solid; MS (ISP): 204.9 [M-H⁻].

### c) N-[2-Methyl-1-(3-phenyl-1,2,4-oxadiazol-5-yl)propan-2-yl]-6-(2-oxopyrrolidin-1-yl)pyridine-2-carboxamide

The title compound was synthesized in analogy to Example 1d, using 6-(2-oxopyrrolidin-1-yl)pyridine-2-carboxylic acid (Example 12b, 0.1 mmol) and α,α-dimethyl-3-phenyl-1,2,4-oxadiazole-5-ethanamine hydrochloride (1:1) (CAN 1426444-03-2, 0.1 mmol) as starting materials and isolated (13 mg, 28%) as colorless oil, LC-MS (UV peak area/ESI) 100%, 406.1874 [MH⁺].

### Example 13

### 6-(3-Chlorophenyl)-N-[1-(5-cyclobutyl-1,3,4-oxadiazol-2-yl)-2-methylpropan-2-yl]pyridine-2-carboxamide

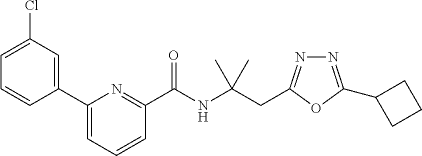 [SMILES: CC(C)(Cc1nnc(C2CCC2)o1)NC(=O)c1cccc(-c2cccc(Cl)c2)n1]

### a) tert-Butyl 4-(2-(cyclobutanecarbonyl)hydrazinyl)-2-methyl-4-oxobutan-2-ylcarbamate

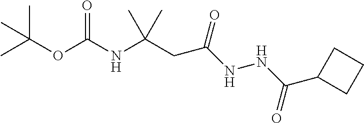 [SMILES: CC(C)(CC(=O)NNC(=O)C1CCC1)NC(=O)OC(C)(C)C]

The title compound was synthesized in analogy to Example 1a, using 3-(tert-butoxycarbonylamino)-3-methylbutanoic acid (CAN 129765-95-3, 3.81 g, 17.5 mmol) and cyclobutanecarbohydrazide (CAN 98069-56-8, 2 g, 17.5 mmol) as starting materials and isolated (4.7 g, 86%) as off-white solid, MS (ISP): 314.2 [MH⁺].

### b) tert-Butyl 1-(5-cyclobutyl-1,3,4-oxadiazol-2-yl)-2-methylpropan-2-ylcarbamate

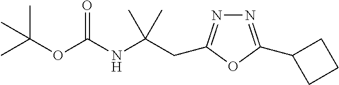 [SMILES: CC(C)(Cc1nnc(C2CCC2)o1)NC(=O)OC(C)(C)C]

The title compound was synthesized in analogy to Example 1b, using tert-butyl 4-(2-(cyclobutanecarbonyl)hydrazinyl)-2-methyl-4-oxobutan-2-ylcarbamate (Example 13a, 4.7 g, 15 mmol) as starting material and isolated (3.4 g, 77%) as off-white solid, MS (ISP): 296.3 [MH⁺].

### c) 1-(5-Cyclobutyl-1,3,4-oxadiazol-2-yl)-2-methyl-propan-2-amine hydrochloride

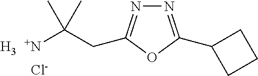

The title compound was synthesized in analogy to Example 1c, using tert-butyl 1-(5-cyclobutyl-1,3,4-oxadiazol-2-yl)-2-methylpropan-2-ylcarbamate (Example 13b, 3.4 g, 11.5 mmol) as starting material and isolated (2.4 g, 90%) as white solid, MS (ISP): 196.3 [MH⁺].

### d) 6-(3-Chlorophenyl)-N-[1-(5-cyclobutyl-1,3,4-oxadiazol-2-yl)-2-methylpropan-2-yl]pyridine-2-carboxamide

The title compound was synthesized in analogy to Example 1d, using 6-(3-chlorophenyl)-2-pyridinecarboxylic acid (CAN 863704-38-5, 64 μmol) and 1-(5-cyclobutyl-1,3,4-oxadiazol-2-yl)-2-methyl-propan-2-amine hydrochloride (77 μmol) as starting materials and isolated (7 mg, 27%) as colorless oil, MS (ISP): 411.3 [MH⁺].

### Example 14

### 6-(2,4-Dichlorophenyl)-N-[2-(5-phenyl-1,3,4-oxadiazol-2-yl)propan-2-yl]pyridine-2-carboxamide

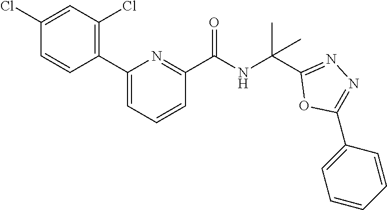 [SMILES: CC(C)(NC(=O)c1cccc(-c2ccc(Cl)cc2Cl)n1)c1nnc(-c2ccccc2)o1]

The title compound (7 mg, 70%) was synthesized in analogy to Example 1d, using 6-(2,4-dichlorophenyl)-2-pyridinecarboxylic acid (CAN 1261912-00-8, 22 μmol) and α,α-dimethyl-5-phenyl-1,3,4-oxadiazole-2-methanamine (CAN 68176-04-5, 24 μmol) as starting materials, LC-MS (EI): 453.0 [MH⁺]. ¹H NMR (300 MHz, CDCl₃): δ 8.66 (bs, 1H), 8.13 (dd, 1H, J₁=7.5 Hz, J₂=0.9 Hz), 8.04-8.00 (m, 2H), 7.93 (t, 1H, J=7.8 Hz), 7.78 (dd, 1H, J₁=7.8 Hz, J₂=0.9 Hz), 7.62-7.40 (m, 6H), 1.98 (s, 6H).

### Example 15

### 6-(2,4-Dichlorophenyl)-N-[2-(5-methyl-1,3,4-oxadiazol-2-yl)propan-2-yl]pyridine-2-carboxamide

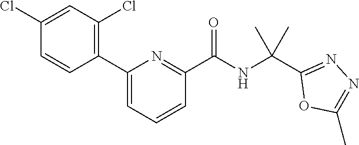 [SMILES: Cc1nnc(C(C)(C)NC(=O)c2cccc(-c3ccc(Cl)cc3Cl)n2)o1]

The title compound (2 mg, 23%) was synthesized in analogy to Example 1d, using 6-(2,4-dichlorophenyl)-2-pyridinecarboxylic acid (CAN 1261912-00-8, 22 μmol) and α,α,5-trimethyl-1,3,4-oxadiazole-2-methanamine (CAN 1368716-09-9, 24 μmol) as starting materials, LC-MS (EI): 390.7 [MH⁺]. ¹H NMR (300 MHz, CDCl₃): δ 8.56 (bs, 1H), 8.13 (dd, 1H, J₁=7.8 Hz, J₂=0.9 Hz), 7.93 (t, 1H, J=7.8 Hz), 7.78 (dd, 1H, J₁=8.1 Hz, J₂=1.2 Hz), 7.59-7.54 (m, 2H), 7.41 (dd, 1H, J₁=7.8 Hz, J₂=1.8 Hz), 2.52 (s, 3H), 1.89 (s, 6H).

### Example 16

### 6-(Cyclopropylmethoxy)-5-(3,3-difluoroazetidin-1-yl)-N-[3-[1-(2-methoxyethoxy)-2-methylpropan-2-yl]-1,2-oxazol-5-yl]pyridine-2-carboxamide

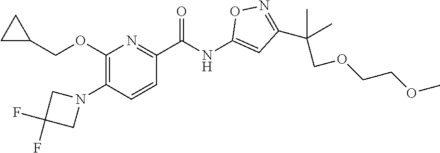 [SMILES: COCCOCC(C)(C)c1cc(NC(=O)c2ccc(N3CC(F)(F)C3)c(OCC3CC3)n2)on1]

The title compound (20 mg, 12%) was synthesized in analogy to Example 1d, using 6-(cyclopropylmethoxy)-5-(3,3-difluoroazetidin-1-yl)pyridine-2-carboxylic acid (CAN 1415898-88-2, 351 μmol) and 3-[2-(2-methoxyethoxy)-1,1-dimethyl-ethyl]isoxazol-5-amine (CAN 1218915-72-0, 383 μmol) as starting materials, LC-MS (EI): 481.2 [MH⁺]. ¹H NMR (300 MHz, CD₃OD): δ 7.68 (d, 1H, J=7.8 Hz), 6.80 (d, 1H, J=7.8 Hz), 6.45 (s, 1H), 4.44 (t, 4H, J=12.0 Hz), 4.29 (d, 2H, J=7.2Hz), 3.60-3.50 (m, 6H), 3.34 (s, 3H), 1.40-1.25 (m, 7H), 0.70-0.60 (m, 2H), 0.45-0.38 (m, 2H).

### Example 17

### 5-Cyclopropyl-6-(cyclopropylmethoxy)-N-[1-(1-hydroxy-2-methylpropan-2-yl)pyrazol-4-yl]pyridine-2-carboxamide

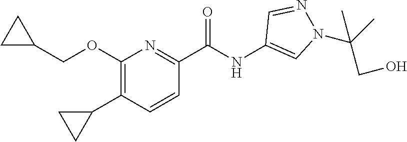 [SMILES: CC(C)(CO)n1cc(NC(=O)c2ccc(C3CC3)c(OCC3CC3)n2)cn1]

### a) 2-(4-Aminopyrazol-1-yl)-2-methyl-propan-1-ol

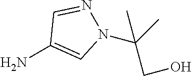

To a solution of 5-hydroxy-4,4-dimethyl-3-oxo-pentanenitrile (CAN 489432-33-9, 5 g, 35 mmol) and NaOH (2.6 g, 65 mmol) in water (100 mL) is added NH₂OH.HCl (2.8 g, 41 mmol). The mixture is heated to 100° C. for 12 hours. After this time, the reaction mixture was cooled to room temperature and extracted with EtOAc (3×150 mL), the organic layers were combined and washed with brine, dried over Na₂SO₄, filtered and concentrated under reduced pressure. The residue was purified by column chromatography to give the title compound (2 g, 36%) as yellow solid, LC-MS (EI): 157.2 [MH⁺].

### b) 5-Cyclopropyl-6-(cyclopropylmethoxy)-N-[1-(1-hydroxy-2-methylpropan-2-yl)pyrazol-4-yl]pyridine-2-carboxamide

The title compound (102 mg, 80%) was synthesized in analogy to Example 1d, using 5-cyclopropyl-6-(cyclopropylmethoxy)pyridine-2-carboxylic acid (CAN 1415898-71-3, 342 μmol) and 2-(4-aminopyrazol-1-yl)-2-methyl-propan-1-ol (373 μmol) as starting materials, LC-MS: 371.2 [MH⁺]. ¹H NMR (300 MHz, CD₃OD): δ 8.18 (s, 1H), 7.81 (s, 1H), 7.63 (d, 1H, J=7.5 Hz), 7.35 (d, 1H, J=7.5 Hz), 4.38 (d, 2H, J=7.8 Hz), 3.74 (s, 2H), 2.25-2.15 (m, 1H), 1.57 (s, 6H), 1.40-1.20 (m, 1H), 1.05-0.43 (m, 8H).

### Example 18

### 5-Cyclopropyl-6-(cyclopropylmethoxy)-N-[3-[1-(2-methoxyethoxy)-2-methylpropan-2-yl]-1,2-oxazol-5-yl]pyridine-2-carboxamide

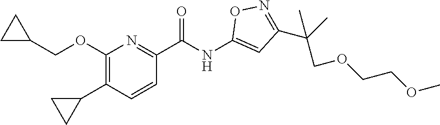 [SMILES: COCCOCC(C)(C)c1cc(NC(=O)c2ccc(C3CC3)c(OCC3CC3)n2)on1]

The title compound (20 mg, 11%) was synthesized in analogy to Example 1d, using 5-cyclopropyl-6-(cyclopropylmethoxy)pyridine-2-carboxylic acid (CAN 1415898-71-3, 429 μmol) and 3-[2-(2-methoxyethoxy)-1,1-dimethyl-ethyl]isoxazol-5-amine (CAN 1218915-72-0, 468 μmol) as starting materials, LC-MS: 430.2 [MH⁺]. ¹H NMR (300 MHz, CD₃OD): δ 7.67 (dd, 1H, J₁=7.5 Hz, J₂=0.3 Hz), 7.35 (d, 1H, J=7.2 Hz), 6.49 (s, 1H), 4.36 (d, 2H, J=7.2 Hz), 3.60-3.50 (m, 6H), 3.34-3.30 (m, 3H), 2.25-2.15 (m, 1H), 1.40-1.20 (m, 7H), 1.08-1.02 (m, 2H), 0.83-0.78 (m, 2H), 0.67-0.61 (m, 2H), 0.46-0.43 (m, 2H).

### Example 19

### 5-Cyclopropyl-6-(cyclopropylmethoxy)-N-[3-(1-hydroxy-2-methylpropan-2-yl)-1,2-oxazol-5-yl]pyridine-2-carboxamide

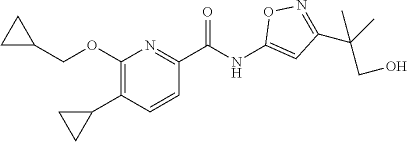 [SMILES: CC(C)(CO)c1cc(NC(=O)c2ccc(C3CC3)c(OCC3CC3)n2)on1]

The title compound (25 mg, 16%) was synthesized in analogy to Example 1d, using 5-cyclopropyl-6-(cyclopropylmethoxy)pyridine-2-carboxylic acid (CAN 1415898-71-3, 429 μmol) and 2-(5-aminoisoxazol-3-yl)-2-methyl-propan-1-ol (CAN 1188910-70-4, 468 μmol) as starting materials, LC-MS: 372.1 [MH⁺]. ¹H NMR (300 MHz, CD₃OD): δ 7.59 (d, 1H, J=7.8 Hz), 7.29 (d, 1H, J=7.8 Hz), 6.39 (s, 1H), 4.28 (d, 2H, J=7.2 Hz), 3.51 (s, 2H), 2.15-2.10 (m, 1H), 1.30-1.10 (m, 7H), 1.00-0.93 (m, 2H), 0.75-0.69 (m, 2H), 0.59-0.52 (m, 2H), 0.36-0.33 (m, 2H).

### Example 20

### 5-Cyclopropyl-6-(cyclopropylmethoxy)-N-[3-[1-(2-ethoxyethoxy)-2-methylpropan-2-yl]-1,2-oxazol-5-yl]pyridine-2-carboxamide

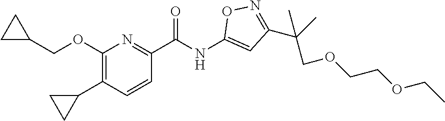 [SMILES: CCOCCOCC(C)(C)c1cc(NC(=O)c2ccc(C3CC3)c(OCC3CC3)n2)on1]

The title compound (25 mg, 13%) was synthesized in analogy to Example 1d, using 5-cyclopropyl-6-(cyclopropylmethoxy)pyridine-2-carboxylic acid (CAN 1415898-71-3, 429 μmol) and 3-[2-(2-ethoxyethoxy)-1,1-dimethyl-ethyl]isoxazol-5-amine (CAN 1218915-74-2, 468 μmol) as starting materials, LC-MS: 444.3 [MH⁺]. ¹H NMR (300 MHz, CD₃OD): δ 7.54 (d, 1H, J=7.5 Hz), 7.23 (d, 1H, J=7.5 Hz), 6.39 (s, 1H), 4.24 (d, 2H, J=6.9 Hz), 3.48-3.36 (m, 8H), 2.20-2.00 (m, 1H), 1.30-1.05 (m, 7H), 1.05 (t, 3H, J=7.2 Hz), 0.96-0.91 (m, 2H), 0.72-0.67 (m, 2H), 0.56-0.50 (m, 2H), 0.35-0.30 (m, 2H).

### Example 21

### 6-(Cyclopropylmethoxy)-5-(3,3-difluoroazetidin-1-yl)-N-(3-hydroxy-1-adamantyl)pyridine-2-carboxamide

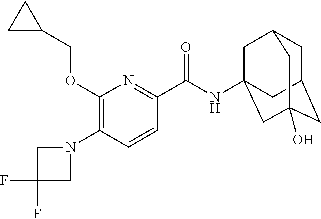 [SMILES: O=C(NC12CC3CC(CC(O)(C3)C1)C2)c1ccc(N2CC(F)(F)C2)c(OCC2CC2)n1]

The title compound (8 mg, 53%) was synthesized in analogy to Example 1d, using 6-(cyclopropylmethoxy)-5-(3,3-difluoroazetidin-1-yl)pyridine-2-carboxylic acid (CAN 1415898-88-2, 35 μmol) and 3-aminoadamantan-1-ol (CAN 702-82-9, 42 μmol) as starting materials and isolated as white solid, MS (ESI): 434.5 [MH⁺].

### Example 22

### tert-Butyl 2-[[[5-cyclopropyl-6-[(4-fluorophenyl)methyl]pyridine-2-carbonyl]amino]methyl]morpholine-4-carboxylate

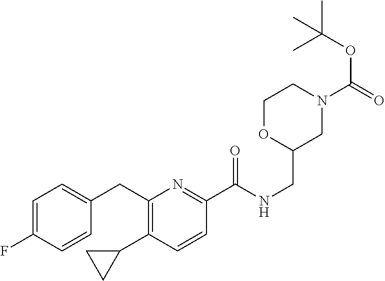 [SMILES: CC(C)(C)OC(=O)N1CCOC(CNC(=O)c2ccc(C3CC3)c(Cc3ccc(F)cc3)n2)C1]

The title compound (132 mg, 51%) was synthesized in analogy to Example 1d, using 5-cyclopropyl-6-[(4-fluorophenyl)methyl]pyridine-2-carboxylic acid (CAN 1415899-48-7, 553 μmol) and tert-butyl 2-(aminomethyl)morpholine-4-carboxylate (CAN 140645-53-0, 664 μmol) as starting materials and isolated as colorless oil, MS (ESI): 470.5 [MH⁺].

### Example 23

### (+)-4-[5-Cyclopropyl-6-(cyclopropylmethoxy)pyrazine-2-carbonyl]thiomorpholine-3-carboxamide

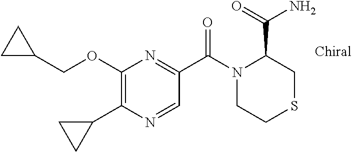 [SMILES: NC(=O)[C@H]1CSCCN1C(=O)c1cnc(C2CC2)c(OCC2CC2)n1]

### a) 5-Bromo-3-cyclopropylmethoxy-pyrazin-2-ylamine

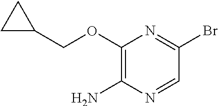 [SMILES: Nc1ncc(Br)nc1OCC1CC1]

To a solution of cyclopropyl-methanol (16.47 mL, 205.62 mmol) in dimethyl sulfoxide (200 mL) was added sodium hydride (60% in oil, 4.93 g, 205.62 mmol) at 0° C. and the reaction mixture was stirred at 0° C. for 2 hours. To this suspension was added 3,5-dibromo-pyrazin-2-ylamine (20 g, 79.09 mmol) in dimethyl sulfoxide (40 mL) and the mixture was stirred at ambient temperature for 12 hours. The mixture was partitioned between water (300 mL) and ethyl acetate and the organic phase was dried with Na₂SO₄, filtered and concentrated in vacuo. The crude material was purified by chromatography (silica gel, 500 g, 10% ethyl acetate in hexane) to give the desired product (14 g, 72.52%) as yellow solid; LC-MS (UV peak area, ESI) 94.69%, 244.0 [MH⁺].

### b) Di-tert-butyl[5-bromo-3-(cyclopropylmethoxy)pyrazin-2-yl]imidodicarbonate

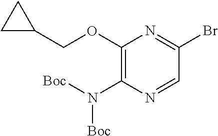 [SMILES: CC(C)(C)OC(=O)N(C(=O)OC(C)(C)C)c1ncc(Br)nc1OCC1CC1]

To a solution of 5-bromo-3-cyclopropylmethoxy-pyrazin-2-ylamine (30 g, 122.91 mmol) in dichloromethane (200 mL) were added di-tert-butyl dicarbonate (67.7 mL, 307.26 mmol) and 4-dimethylaminopyridine (1.49 g, 12.29 mmol). The reaction mixture was stirred at ambient temperature for 18 hours. The mixture was partitioned between water (300 mL) and dichloromethane and the organic phase was separated, washed with brine, dried with Na₂SO₄, filtered and concentrated in vacuo. The crude material was purified by chromatography (silica gel, 600 g, 5%-7% ethyl acetate in hexane) to give the desired product (45 g, 82.77%) as yellow oil; LC-MS (UV peak area, ESI) 94.69%, 445.0 [MH⁺].

### c) Methyl 5-[bis(tert-butoxycarbonyl)amino]-6-(cyclopropylmethoxy)pyrazine-2-carboxylate

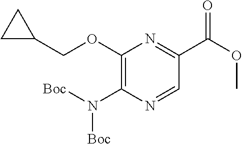 [SMILES: COC(=O)c1cnc(N(C(=O)OC(C)(C)C)C(=O)OC(C)(C)C)c(OCC2CC2)n1]

To a solution of di-tert-butyl[5-bromo-3-(cyclopropylmethoxy)pyrazin-2-yl]imido-dicarbonate (20 g, 45.05 mmol) in methanol (200 mL) was added PdCl₂.dppf.CH₂Cl₂ (4.04 g, 4.95 mmol) and triethylamine (9.5 mL, 67.57 mmol) and the mixture was stirred under an atmosphere of 32 bar carbon monoxide at 80° C. for 5 hours. After expansion and cooling, the solid was removed by filtration. The organic phase was separated, washed with brine (300 mL), dried with Na₂SO₄, filtered and concentrated in vacuo. The crude material was purified by chromatography (Combi-Flash®, 120 g, 15%-20% ethyl acetate in hexane) to give the desired product (14 g, 73.68%) as yellow semi-solid; LC-MS (UV peak area, ESI) 96.14%, 424.4 [MH⁺].

### d) 5-Amino-6-cyclopropylmethoxy-pyrazine-2-carboxylic acid methyl ester

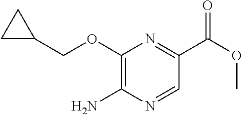 [SMILES: COC(=O)c1cnc(N)c(OCC2CC2)n1]

Methyl 5-[bis(tert-butoxycarbonyl)amino]-6-(cyclopropylmethoxy)pyrazine-2-carboxylate (15 g, 35.46 mmol) was suspended in methanol (150 mL) and water (225 mL) and the mixture was heated at 100° C. for 12 hours. After cooling, white solid was formed, filtered and dried in vacuo to give the title compound (5.7 g, 72.15%) as off white solid; LC-MS (UV peak area, ESI) 99.68%, 224.2 [MH⁺].

### e) 5-Bromo-6-cyclopropylmethoxy-pyrazine-2-carboxylic acid methyl ester

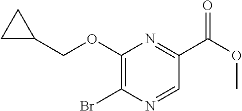 [SMILES: COC(=O)c1cnc(Br)c(OCC2CC2)n1]

5-Amino-6-cyclopropylmethoxy-pyrazine-2-carboxylic acid methyl ester (10 g, 44.84 mmol) was suspended in dibromomethane (150 mL). To this suspension were added trimethylsilyl bromide (14.8 mL, 112.11 mmol) followed by tert-butyl nitrite (57.5 mL, 448.43 mmol) at 0° C. and the mixture was stirred at that temperature for 3 hours. The mixture was partitioned between water (190 mL) and ethyl acetate and the organic phase was washed with brine (200 mL), dried with Na₂SO₄, filtered and concentrated in vacuo. The crude material was purified by chromatography (Combi-Flash, 80 g, 20% ethyl acetate in hexane) to give the desired product (6.3 g, 46.6%) as white solid; LC-MS (UV peak area, ESI) 90.68%, 287.2 [MH⁺].

### f) 5-Cyclopropyl-6-cyclopropylmethoxy-pyrazine-2-carboxylic acid methyl ester

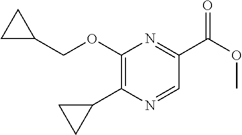 [SMILES: COC(=O)c1cnc(C2CC2)c(OCC2CC2)n1]

5-Bromo-6-cyclopropylmethoxy-pyrazine-2-carboxylic acid methyl ester (5 g, 17.42 mmol), potassium phosphate tribasic (12.9 g, 60.98 mmol) and palladium(II)acetate (389 mg, 1.74 μmol) were dissolved in toluene (45 mL) and water (5 mL) and the reaction mixture was degassed with argon for 15 minutes. Cyclopropylboronic acid (2.9 g, 34.84 mmol) and tricyclohexylphosphine (0.487 g, 1.74 mmol) were added and the reaction mixture was stirred at 60° C. for 16 hours. The mixture was partitioned between water and ethyl acetate and the organic phase was washed with brine (100 mL), dried with Na₂SO₄, filtered and concentrated in vacuo. The crude material was purified by chromatography (Combi-Flash, 80 g, 10%-15% ethyl acetate in hexane) to give the desired product (2.6 g, 60.11%) as white solid; LC-MS (UV peak area, ESI) 98.87%, 249.2 [MH⁺].

### g) 5-Cyclopropyl-6-cyclopropylmethoxy-pyrazine-2-carboxylic acid

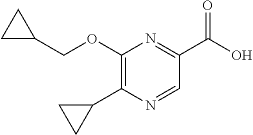 [SMILES: O=C(O)c1cnc(C2CC2)c(OCC2CC2)n1]

To a solution of 5-cyclopropyl-6-cyclopropylmethoxy-pyrazine-2-carboxylic acid methyl ester (7 g, 28.23 mmol) in THF (20 mL) and H₂O (10 mL) was added lithium hydroxide (1.54 g, 26.69 mmol) and the mixture was stirred at ambient temperature for 4.5 hours. Solvent was concentrated in vacuo and residue was diluted with H₂O (20 mL). The aqueous phase was acidified with hydrochloric acid (1M, pH˜2-3) and the solid was separated. The solid was triturated with toluene (25 ml) and dried in vacuo to give the title compound (5.3 g, 86.6%) as white crystalline solid; LC-MS (UV peak area, ESI) 93.2%, 233.2 [M-H⁻].

### h) 4-[5-Cyclopropyl-6-(cyclopropylmethoxy)pyrazine-2-carbonyl]thiomorpholine-3-carboxamide

The title compound was synthesized in analogy to Example 1d, using 5-cyclopropyl-6-cyclopropylmethoxy-pyrazine-2-carboxylic acid (Example 23g, 0.43 mmol) and 3-thiomorpholinecarboxamide (CAN 103742-31-0, 0.43 mmol) as starting materials and isolated (134 mg, 87%) as light yellow solid, LC-MS (UV peak area/ESI) 100%, 363.1490 [MH⁺].

### i) (+)-4-[5-Cyclopropyl-6-(cyclopropylmethoxy)pyrazine-2-carbonyl]thiomorpholine-3-carboxamide

Racemic 4-[5-cyclopropyl-6-(cyclopropylmethoxy)pyrazine-2-carbonyl]thiomorpholine-3-carboxamide (Example 23h, 108 mg) was subjected to chiral chromatography (Reprosil chiral NR, 30% ethanol in heptane) to give the title compound (46 mg, 43%) as light yellow solid; LC-MS (UV peak area/ESI) 100%, 363.1490 [MH⁺]; (+) enantiomer, α_{D}²⁰(MeOH)=+41.3°.

### Example 24

### 5-Cyclopropyl-6-(cyclopropylmethoxy)-N-[3-[2-methyl-1-(oxan-2-yloxy)propan-2-yl]-1,2-oxazol-5-yl]pyrazine-2-carboxamide

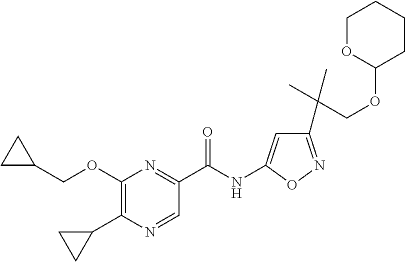 [SMILES: CC(C)(COC1CCCCO1)c1cc(NC(=O)c2cnc(C3CC3)c(OCC3CC3)n2)on1]

To a stirred solution of 5-cyclopropyl-6-cyclopropylmethoxy-pyrazine-2-carboxylic acid (Example 23g, 100 mg, 0.427 mmol) and 3-[1,1-dimethyl-2-[(tetrahydro-2H-pyran-2-yl)oxy]ethyl]-5-isoxazolamine (CAN 1218915-54-8, 153.34 mg, 0.641 mmol) in pyridine (3 mL) was added POC13 at 0° C. and was stirred for 3 hours at room temperature. After completion of the reaction, the reaction mixture was evaporated in vacuo, diluted with ethyl acetate and washed with water. The organic phase was dried over sodium sulfate, filtered and concentrated in vacuo. The residue was purified by silica column chromatography using 20% ethyl acetate in hexane to give the title compound (110 mg, 56%) as light yellow solid; LC-MS (UV peak area, ESI) 98.4%, 457.2 [MH⁺].

### Example 25

### 5-Cyclopropyl-6-(cyclopropylmethoxy)-N-[3-(1-hydroxy-2-methylpropan-2-yl)-1,2-oxazol-5-yl]pyrazine-2-carboxamide

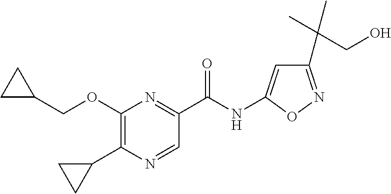 [SMILES: CC(C)(CO)c1cc(NC(=O)c2cnc(C3CC3)c(OCC3CC3)n2)on1]

To a stirred solution of 5-cyclopropyl-6-(cyclopropylmethoxy)-N-[3-[2-methyl-1-(oxan-2-yloxy)propan-2-yl]-1,2-oxazol-5-yl]pyrazine-2-carboxamide (Example 24, 1.0 g, 1.972 mmol) in ethanol (20 mL) was added pyridinium p-toluenesulfonate (0.149 g, 0.592 mmol) and the mixture heated to 70° C. for 1 hour. After completion of the reaction, the solvent was removed in vacuo. The residue was purified by silica column chromatography using 30% ethyl acetate in hexane to give the title compound (600 mg, 82%) as white solid; LC-MS (UV peak area, ESI) 98.9%, 373.0 [MH⁺].

### Example 26

### N-[3-(1-Azido-2-methylpropan-2-yl)-1,2-oxazol-5-yl]-5-cyclopropyl-6-(cyclopropylmethoxy)pyrazine-2-carboxamide

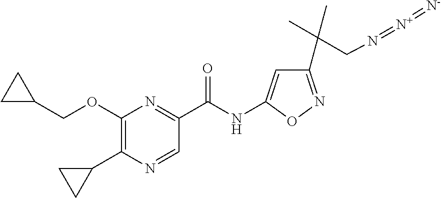 [SMILES: CC(C)(CN=[N+]=[N-])c1cc(NC(=O)c2cnc(C3CC3)c(OCC3CC3)n2)on1]

### a) [2-[5-[[5-Cyclopropyl-6-(cyclopropylmethoxy)pyrazine-2-carbonyl]amino]-1,2-oxazol-3-yl]-2-methylpropyl] methanesulfonate

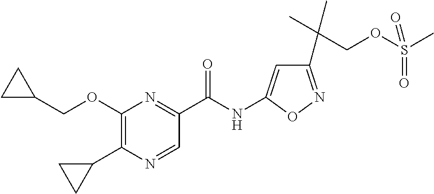 [SMILES: CC(C)(COS(C)(=O)=O)c1cc(NC(=O)c2cnc(C3CC3)c(OCC3CC3)n2)on1]

To a stirred solution 5-cyclopropyl-6-(cyclopropylmethoxy)-N-[3-(1-hydroxy-2-methylpropan-2-yl)-1,2-oxazol-5-yl]pyrazine-2-carboxamide (Example 25, 400 mg, 1.075 mmol) in DCM (15 mL) were added triethylamine (0.724 mL, 5.376 mmol) and mesyl chloride (0.166 mL, 2.151mmol) at 0° C. The reaction mixture was stirred at room temperature for 2 hours. After completion of the reaction, the reaction mixture was diluted with DCM and washed with aqueous saturated sodium bicarbonate solution. The organic phase was dried over sodium sulfate, filtered and concentrated in vacuo. The residue consisted mostly of the title compound (450 mg) as a brown sticky liquid which was directly used for next step; LC-MS (UV peak area, ESI) 93.5%, 451.1 [MH⁺].

### b) N-[3-(1-Azido-2-methylpropan-2-yl)-1,2-oxazol-5-yl]-5-cyclopropyl-6-(cyclopropylmethoxy)pyrazine-2-carboxamide

To a stirred solution of [2-[5-[[5-cyclopropyl-6-(cyclopropylmethoxy)pyrazine-2-carbonyl]amino]-1,2-oxazol-3-yl]-2-methylpropyl] methanesulfonate (Example 26a, 200 mg, crude) in DMF (2 mL) was added sodium azide (144.4 mg, 2.22 mmol) and heated to 120° C. in sealed tube for 16 hours. After completion of the reaction, the reaction mixture was cooled to room temperature then diluted with ethyl acetate and washed with water. The organic phase was dried over sodium sulfate and concentrated in vacuo. The residue was purified by 1 silica column chromatography using 20% ethyl acetate in hexane to give the title compound (60 mg, 32% after two steps) as white solid; LC-MS (UV peak area, ESI) 99.7%, 398.2 [MH⁺].

### Example 27

### 6-(Cyclopropylmethoxy)-5-(3,3-difluoroazetidin-1-yl)-N-[1-[5-(4-fluorophenyl)-1,3,4-oxadiazol-2-yl]-2-methylpropan-2-yl]pyridine-2-carboxamide

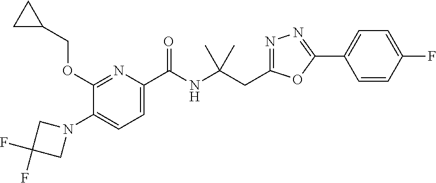 [SMILES: CC(C)(Cc1nnc(-c2ccc(F)cc2)o1)NC(=O)c1ccc(N2CC(F)(F)C2)c(OCC2CC2)n1]

### a) tert-Butyl 4-(2-(4-fluorobenzoyl)hydrazinyl)-2-methyl-4-oxobutan-2-ylcarbamate

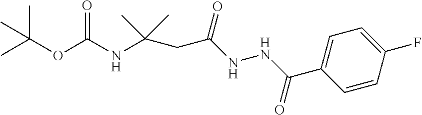 [SMILES: CC(C)(CC(=O)NNC(=O)c1ccc(F)cc1)NC(=O)OC(C)(C)C]

The title compound was synthesized in analogy to Example 1a, using 3-(tert-butoxycarbonylamino)-3-methylbutanoic acid (CAN 129765-95-3, 3 g, 13.8 mmol) and 4-fluorobenzohydrazide (CAN 456-06-4, 2.1 g, 13.8 mmol) as starting materials and isolated (1.3 g, 26%) as yellow oil, MS (ISP): 354.3 [MH⁺].

### b) tert-butyl 1-(5-(4-fluorophenyl)-1,3,4-oxadiazol-2-yl)-2-methylpropan-2-ylcarbamate

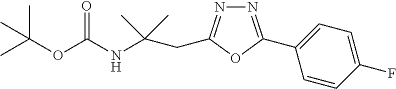 [SMILES: CC(C)(Cc1nnc(-c2ccc(F)cc2)o1)NC(=O)OC(C)(C)C]

The title compound was synthesized in analogy to Example 1b, using tert-butyl 4-(2-(4-fluorobenzoyl)hydrazinyl)-2-methyl-4-oxobutan-2-ylcarbamate (Example 27a, 1.3 g, 3.7 mmol) as starting material and isolated (0.99 g, 81%) as white solid, MS (ISP): 336.3 [MH⁺].

### c) 1-(5-(4-Fluorophenyl)-1,3,4-oxadiazol-2-yl)-2-methylpropan-2-amine hydrochloride

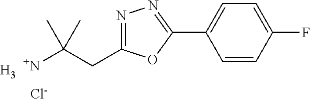

The title compound was synthesized in analogy to Example 1c, using, tert-butyl 1-(5-(4-fluorophenyl)-1,3,4-oxadiazol-2-yl)-2-methylpropan-2-ylcarbamate (Example 27b, 0.98 g, 2.9 mmol) as starting material and isolated (620 mg, 78%), MS (ESI): 236.2 [MH⁺].

### d) 6-(Cyclopropylmethoxy)-5-(3,3-difluoroazetidin-1-yl)-N-[1-[5-(4-fluorophenyl)-1,3,4-oxadiazol-2-yl]-2-methylpropan-2-yl]pyridine-2-carboxamide

To a solution of 6-(cyclopropylmethoxy)-5-(3,3-difluoroazetidin-1-yl)pyridine-2-carboxylic acid (CAN 1415898-88-2, 20 mg, 70.4 μmol) in dichloromethane (1 mL) was added DIPEA (22.7 mg, 30.7 μL, 176 μmol) and 4-(4,6-dimethoxy-1,3,5-triazin-2-yl)-4-methylmorpholin-4-ium chloride (21.4 mg, 77.4 μmol). The mixture was stirred for 30 min at ambient temperature, then 1-(5-(4-fluorophenyl)-1,3,4-oxadiazol-2-yl)-2-methylpropan-2-amine hydrochloride (Example 27c, 16.6 mg, 70.4 μmol) was added. The reaction mixture was stirred at ambient temperature overnight, diluted with dichloromethane (8 mL) and washed with 1 M aq. NaHCO₃ solution (3×10 mL), water (10 mL) and brine (15 mL). The organic phase was dried over MgSO₄ and concentrated under reduced pressure. Flash chromatography (10 g SiO₂, heptane/EtOAc 4:1 to 1:1) gave the title compound (19.7 mg, 56%), MS (ESI): 502.6 [MH⁺].

### Example 28

### 5-Cyclopentyl-6-(cyclopropylmethoxy)-N-[1-[5-(4-fluorophenyl)-1,3,4-oxadiazol-2-yl]-2-methylpropan-2-yl]pyridine-2-carboxamide

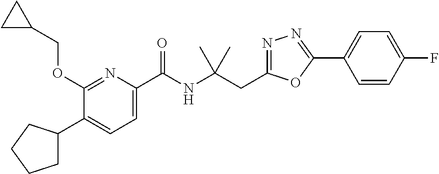 [SMILES: CC(C)(Cc1nnc(-c2ccc(F)cc2)o1)NC(=O)c1ccc(C2CCCC2)c(OCC2CC2)n1]

The title compound was synthesized in analogy to the procedure described in Example 27d, using 5-cyclopentyl-6-(cyclopropylmethoxy)pyridine-2-carboxylic acid (CAN 1415898-70-2, 20 mg, 77 μmol) and 1-(5-(4-fluorophenyl)-1,3,4-oxadiazol-2-yl)-2-methylpropan-2-amine hydrochloride (Example 27c, 23 mg, 85 μmol) and isolated (18 mg, 49%), LC-MS (ESI): 479.7 [MH⁺].

### Example 29

### 5-Cyclopentyl-6-(cyclopropylmethoxy)-N-[3-[1-(2-methoxyethoxy)-2-methylpropan-2-yl]-1,2-oxazol-5-yl]pyridine-2-carboxamide

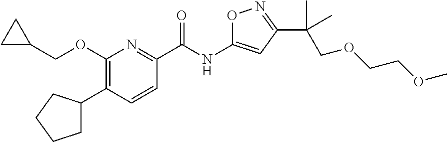 [SMILES: COCCOCC(C)(C)c1cc(NC(=O)c2ccc(C3CCCC3)c(OCC3CC3)n2)on1]

The title compound was synthesized in analogy to the procedure described in Example 27d, using 5-cyclopentyl-6-(cyclopropylmethoxy)pyridine-2-carboxylic acid (CAN 1415898-70-2, 21 mg, 82 μmol) and 3-[2-(2-methoxyethoxy)-1,1-dimethyl-ethyl]isoxazol-5-amine (CAN 1218915-72-0, 18 mg, 82 μmol) and isolated (10 mg, 26%) as colorless oil, LC-MS (ESI): 458.7 [MH⁺].

Example 30

### 5-Cyclopentyl-6-(cyclopropylmethoxy)-N-[3-[1-(2-ethoxyethoxy)-2-methylpropan-2-yl]-1,2-oxazol-5-yl]pyridine-2-carboxamide

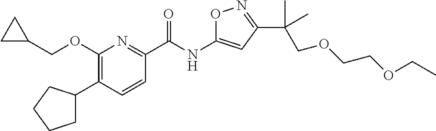 [SMILES: CCOCCOCC(C)(C)c1cc(NC(=O)c2ccc(C3CCCC3)c(OCC3CC3)n2)on1]

The title compound was synthesized in analogy to the procedure described in Example 27d, using 5-cyclopentyl-6-(cyclopropylmethoxy)pyridine-2-carboxylic acid (CAN 1415898-70-2, 22 mg, 83 μmol) and 3-[2-(2-ethoxyethoxy)-1,1-dimethyl-ethyl]isoxazol-5-amine (CAN 1218915-74-2, 19 mg, 83 μmol) and isolated (10 mg, 25%), LC-MS (ESI): 472.8 [MH⁺].

### Example 31

### Pharmacological Tests

The following tests were carried out in order to determine the activity of the compounds of formula I:

Radioligand Binding Assay

The affinity of the compounds of the invention for cannabinoid CB1 receptors was determined using recommended amounts of membrane preparations (PerkinElmer) of human embryonic kidney (HEK) cells expressing the human CNR1 or CNR2 receptors in conjunction with 1.5 or 2.6 nM [3H]-CP-55,940 (Perkin Elmer) as radioligand, respectively. Binding was performed in binding buffer (50 mM Tris, 5 mM MgCl2, 2.5 mM EDTA, and 0.5% (wt/vol) fatty acid free BSA, pH 7.4 for CB1 receptor and 50 mM Tris, 5 mM MgCl2, 2.5 mM EGTA, and 0.1% (wt/vol) fatty acid free BSA, pH 7.4 for CB2 receptor) in a total volume of 0.2 ml for 1 h at 30° C. shaking. The reaction was terminated by rapid filtration through microfiltration plates coated with 0.5% polyethylenimine (UniFilter GF/B filter plate; Packard). Bound radioactivity was analyzed for Ki using nonlinear regression analysis (Activity Base, ID Business Solution, Limited), with the Kd values for [3H]CP55,940 determined from saturation experiments. The compounds of formula (I) show an excellent affinity for the CB2 receptor.

The compounds according to formula (I) have an activity in the above assay (Ki) between 0.5 nM and 10 μM. Particular compounds of formula (I) have an activity in the above assay (Ki) between 0.5 nM and 3 μM. Other particular compounds of formula (I) have an activity in the above assay (Ki) between 0.5 nM and 100 nM.

cAMP Assay

CHO cells expressing human CB1 or CB2 receptors are seeded 17-24 hours prior to the experiment 50.000 cells per well in a black 96 well plate with flat clear bottom (Corning Costar #3904) in DMEM (Invitrogen No. 31331), 1× HT supplement, with 10% fetal calf serum and incubated at 5% CO₂ and 37° C. in a humidified incubator. The growth medium was exchanged with Krebs Ringer Bicarbonate buffer with 1 mM IBMX and incubated at 30° C. for 30 min. Compounds were added to a final assay volume of 100 μl and incubated for 30 min at 30° C. Using the cAMP-Nano-TRF detection kit the assay (Roche Diagnostics) was stopped by the addition of 50 μl lysis reagent (Tris, NaCl, 1.5% Triton X100, 2.5% NP40, 10% NaN₃) and 50 μl detection solutions (20 μM mAb Alexa700-cAMP 1:1, and 48 μM Ruthenium-2-AHA-cAMP) and shaken for 2 h at room temperature. The time-resolved energy transfer is measured by a TRF reader (Evotec Technologies GmbH), equipped with a ND:YAG laser as excitation source. The plate is measured twice with the excitation at 355 nm and at the emission with a delay of 100 ns and a gate of 100 ns, total exposure time 10 s at 730 (bandwidth 30 nm) or 645 nm (bandwidth 75 nm), respectively. The FRET signal is calculated as follows: FRET=T730−Alexa730−P(T645−B645) with P=Ru730−B730/Ru645−B645, where T730 is the test well measured at 730 nM, T645 is the test well measured at 645 nm, B730 and B645 are the buffer controls at 730 nm and 645 nm, respectively. cAMP content is determined from the function of a standard curve spanning from 10 μM to 0.13 nM cAMP.

EC₅₀ values were determined using Activity Base analysis (ID Business Solution, Limited). The EC₅₀ values for a wide range of cannabinoid agonists generated from this assay for reference compounds were in agreement with the values published in the scientific literature.

In the foregoing assay, the compounds according to the invention have a human CB2 EC₅₀ which is between 0.5 nM and 10 μM. Particular compounds according to the invention have a human CB2 EC₅₀ between 0.5 nM and 1 μM. Further particular compounds according to the invention have a human CB2 EC₅₀ between 0.5 nM and 100 nM. They exhibit at least 10 fold selectivity against the human CB1 receptor in, either both of the radioligand and cAMP assay, or in one of these two assays.

Results obtained for representative compounds of the invention are given in the following table.

| | cAMP assay |
|---|---|
| Example | human CB2 EC₅₀ [μM] |
| 1 | 0.4965 |
| 2 | 0.1646 |
| 3 | 0.1321 |
| 4 | 0.3394 |
| 5 | 0.5537 |
| 6 | 0.1822 |
| 7 | 0.2438 |
| 8 | 0.1945 |
| 9 | 0.0367 |
| 10 | 0.2647 |
| 11 | 0.5735 |
| 12 | 0.6621 |
| 13 | 0.2939 |
| 14 | 0.2271 |
| 15 | 0.1070 |
| 16 | 0.2185 |
| 17 | 0.2236 |
| 18 | 0.0352 |
| 19 | 0.1387 |
| 20 | 0.0225 |
| 21 | 0.0816 |
| 22 | 0.1008 |
| 23 | 0.5893 |
| 24 | 0.0752 |
| 25 | 0.0516 |
| 26 | 0.0124 |
| 27 | 2.889 |
| 28 | 2.758 |
| 29 | 0.175 |
| 30 | 0.176 |

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is then mixed with sodium starch glycolate and magnesium stearate and compressed to yield kernels of 120 or 350 mg respectively. The kernels are lacquered with an aq. solution/suspension of the above mentioned film coat.

### Example B

Capsules containing the following ingredients can be manufactured in a conventional manner:

| Ingredients | Per capsule |
|---|---|
| Compound of formula (I) | 25.0 mg |
| Lactose | 150.0 mg |
| Maize starch | 20.0 mg |
| Talc | 5.0 mg |

The components are sieved and mixed and filled into capsules of size 2.

### Example C

Injection solutions can have the following composition:

| | |
|---|---|
| Compound of formula (I) | 3.0 mg |
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene glycol 400 and water for injection (part). The pH is adjusted to 5.0 by addition of acetic acid. The volume is adjusted to 1.0 ml by addition of the residual amount of water. The solution is filtered, filled into vials using an appropriate overage and sterilized.

1. A compound of formula (I): 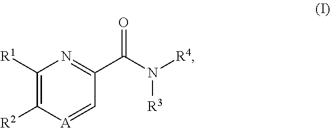
wherein
A is —CH— or nitrogen;
R^{l} is halophenyl, halophenylalkyl, haloalkoxy, halogen, alkoxyalkoxy, oxopyrrolidinyl or cycloalkylalkoxy;
R² is hydrogen, halophenylamino, cycloalkyl or haloazetidinyl;
one of R³ and R⁴ is hydrogen and the other one is —(CR⁵R⁶)ₘ—(CH₂)ₙ—R⁷;
or R³ and R⁴ together with the nitrogen atom to which they are attached form aminocarbonylthiomorpholinyl;
R⁵ and R⁶ are independently selected from hydrogen and alkyl;
R⁷ is 5-cycloalkyl-1,3,4-oxadiazolyl, 3-cycloalkyl-1,2,4-oxadiazolyl, 5-phenyl-1,3,4-oxadiazolyl, 3-phenyl-1,2,4-oxadiyzolyl, 5-alkyl-1,3,4-oxadiazolyl, 3-alkoxyalkoxyalkyl-1,2-oxazolyl, 1-hydroxyalkylpyrazolyl, 3-hydroxy-1-adamantyl, alkoxycarbonylmorpholinyl, 3-oxanyloxyalkyl-1,2-oxazol-5-yl, 3-azidoalkyl-1,2-oxazol-5-yl or 5-(4-fluorophenyl)-1,3,4-oxadiazolyl;
m is 0 or 1; and
n is 0 or 1;
or a pharmaceutically acceptable salt or ester thereof;
provided that the compound is other than 6-chloro-N-(5-cyclopropyl-1,3,4-oxadiazol-2-yl)-2-pyridinecarboxamide and pharmaceutically acceptable salts esters thereof is excluded.

2. The compound according to claim 1, or a pharmaceutically acceptable salt or ester thereof, wherein R¹ is halogen or cycloalkylalkoxy.

3. The compound according to claim 2, or a pharmaceutically acceptable salt or ester thereof, wherein R¹ is chloro or cyclopropylmethoxy.

4. The compound according to claim 1, or a pharmaceutically acceptable salt or ester thereof, wherein R² is halophenylamino or cycloalkyl.

5. The compound according to claim 4, or a pharmaceutically acceptable salt or ester thereof, wherein R² is dichlorophenylamino or cyclopropyl.

6. The compound according to claim 1, or a pharmaceutically acceptable salt or ester thereof, wherein R⁵ and R⁶ are both alkyl at the same time.

7. The compound according to claim 6, or a pharmaceutically acceptable salt or ester thereof, wherein R⁵ and R⁶ are both methyl at the same time.

8. The compound according to claim 1, or a pharmaceutically acceptable salt or ester thereof, wherein R⁷ is 5-phenyl-1,3,4-oxadiyzolyl, 3-alkoxyalkoxyalkyl-1,2-oxazolyl or 3-azidoalkyl-1,2-oxazol-5-yl.

9. The compound according to claim 1, wherein the compound is selected from the group consisting of:
6-(4-chlorophenyl)-N-[1-(5-cyclopropyl-1,3,4-oxadiazol-2-yl)-2-methylpropan-2-yl]pyridine-2-carboxamide;
6-(4-chlorophenyl)-N-[2-methyl-1-(5-phenyl-1,3,4-oxadiazol-2-yl)propan-2-yl]pyridine-2-carboxamide;
N-[2-methyl-1-(5-phenyl-1,3,4-oxadiazol-2-yl)propan-2-yl]-6-(2,2,2-trifluoroethoxy)pyridine-2-carboxamide;
6-(4-chlorophenyl)-N-[2-methyl-1-(3-phenyl-1,2,4-oxadiazol-5-yl)propan-2-yl]pyridine-2-carboxamide;
N-[2-methyl-1-(3-phenyl-1,2,4-oxadiazol-5-yl)propan-2-yl]-6-(2,2,2-trifluoroethoxy)pyridine-2-carboxamide;
6-(3-chlorophenyl)-N-[2-methyl-1-(5-phenyl-1,3,4-oxadiazol-2-yl)propan-2-yl]pyridine-2-carboxamide;
6-(3-chlorophenyl)-N-[1-(3-cyclopropyl-1,2,4-oxadiazol-5-yl)-2-methylpropan-2-yl]pyridine-2-carboxamide;
6-(3-chlorophenyl)-N-[2-methyl-1-(3-phenyl-1,2,4-oxadiazol-5-yl)propan-2-yl]pyridine-2-carboxamide;
6-chloro-5-(2,4-dichloroanilino)-N-[2-methyl-1-(5-phenyl-1,3,4-oxadiazol-2-yl)propan-2-yl]pyridine-2-carboxamide;
6-(4-chlorophenyl)-5-cyclopropyl-N-[2-methyl-1-(5-phenyl-1,3,4-oxadiazol-2-yl)propan-2-yl]pyridine-2-carboxamide;
6-(2-methoxyethoxy)-N-[2-methyl-1-(3-phenyl-1,2,4-oxadiazol-5-yl)propan-2-yl]pyridine-2-carboxamide;
N-[2-methyl-1-(3-phenyl-1,2,4-oxadiazol-5-yl)propan-2-yl]-6-(2-oxopyrrolidin-1-yl)pyridine-2-carboxamide;
6-(3-chlorophenyl)-N-[1-(5-cyclobutyl-1,3,4-oxadiazol-2-yl)-2-methylpropan-2-yl]pyridine-2-carboxamide;
6-(2,4-dichlorophenyl)-N-[2-(5-phenyl-1,3,4-oxadiazol-2-yl)propan-2-yl]pyridine-2-carboxamide;
6-(2,4-dichlorophenyl)-N-[2-(5-methyl-1,3,4-oxadiazol-2-yl)propan-2-yl]pyridine-2-carboxamide;
6-(cyclopropylmethoxy)-5-(3,3-difluoroazetidin-1-yl)-N-[3-[1-(2-methoxyethoxy)-2-methylpropan-2-yl]-1,2-oxazol-5-yl]pyridine-2-carboxamide;
5-cyclopropyl-6-(cyclopropylmethoxy)-N-[1-(1-hydroxy-2-methylpropan-2-yl)pyrazol-4-yl]pyridine-2-carboxamide;
5-cyclopropyl-6-(cyclopropylmethoxy)-N-[3-[1-(2-methoxyethoxy)-2-methylpropan-2-yl]-1,2-oxazol-5-yl]pyridine-2-carboxamide;
5-cyclopropyl-6-(cyclopropylmethoxy)-N-[3-(1-hydroxy-2-methylpropan-2-yl)-1,2-oxazol-5-yl]pyridine-2-carboxamide;
5-cyclopropyl-6-(cyclopropylmethoxy)-N-[3-[1-(2-ethoxyethoxy)-2-methylpropan-2-yl]-1,2-oxazol-5-yl]pyridine-2-carboxamide;
6-(cyclopropylmethoxy)-5-(3,3-difluoroazetidin-1-yl)-N-(3-hydroxy-1-adamantyl)pyridine-2-carboxamide;
tert-butyl 2-[[[5-cyclopropyl-6-[(4-fluorophenyl)methyl]pyridine-2-carbonyl]amino]methyl]morpholine-4-carboxylate;
(3S)-4-[5-cyclopropyl-6-(cyclopropylmethoxy)pyrazine-2-carbonyl]thiomorpholine-3-carboxamide;
5-cyclopropyl-6-(cyclopropylmethoxy)-N-[3-(1-hydroxy-2-methylpropan-2-yl)-1,2-oxazol-5-yl]pyrazine-2-carboxamide;
5-cyclopropyl-6-(cyclopropylmethoxy)-N-[3-[2-methyl -1-(oxan-2-yloxy)propan-2-yl]-1,2-oxazol-5-yl]pyrazine-2-carboxamide;
N-[3-(1-azido-2-methylpropan-2-yl)-1,2-oxazol-5-yl]-5-cyclopropyl-6-(cyclopropylmethoxy)pyrazine-2-carboxamide;
6-(Cyclopropylmethoxy)-5-(3,3-difluoroazetidin-1-yl)-N-[1-[5-(4-fluorophenyl)-1,3,4-oxadiazol-2-yl]-2-methylpropan-2-yl]pyridine-2-carboxamide;
5-Cyclopentyl-6-(cyclopropylmethoxy)-N-[1-[5-(4-fluorophenyl)-1,3,4-oxadiazol-2-yl]-2-methylpropan-2-yl]pyridine-2-carboxamide;
5-Cyclopentyl-6-(cyclopropylmethoxy)-N-[3-[1-(2-methoxyethoxy)-2-methylpropan-2-yl]-1,2-oxazol-5-yl]pyridine-2-carboxamide; and
5-Cyclopentyl-6-(cyclopropylmethoxy)-N-[3-[1-(2-ethoxyethoxy)-2-methylpropan-2-yl]-1,2-oxazol-5-yl]pyridine-2-carboxamide;
or a pharmaceutically acceptable salt thereof.

10. (canceled)

11. (canceled)

12. A pharmaceutical composition comprising a compound in accordance with claim 1, or a pharmaceutically acceptable salt thereof, and a therapeutically inert carrier.

13. A method for the treatment or prophylaxis of pain, neuropathic pain, asthma, osteoporosis, inflammation, psychiatric diseases, psychosis, oncology, encephalitis, malaria, allergy, immunological disorders, arthritis, gastrointestinal disorders, psychiatric disorders rheumatoid arthritis, psychosis or allergy, comprising administering to a patient in need thereof an effective amount of the compound of claim 1, or a pharmaceutically acceptable salt thereof.

14. The compound according to claim 1, wherein the compound is:
6-chloro-5-(2,4-dichloroanilino)-N-[2-methyl-1-(5-phenyl-1,3,4-oxadiazol-2-yl)propan-2-yl]pyridine-2-carboxamide;
or a pharmaceutically acceptable salt thereof.

15. The compound of claim 1, wherein the compound is:
5-cyclopropyl-6-(cyclopropylmethoxy)-N-[3-[1-(2-methoxyethoxy)-2-methylpropan-2-yl]-1,2-oxazol-5-yl]pyridine-2-carboxamide;
or a pharmaceutically acceptable salt thereof.

16. The compound of claim 1, wherein the compound is:
5-cyclopropyl-6-(cyclopropylmethoxy)-N-[3-[1-(2-ethoxyethoxy)-2-methylpropan-2-yl]-1,2-oxazol-5-yl]pyridine-2-carboxamide;
or a pharmaceutically acceptable salt thereof.

17. The compound of claim 1, wherein the compound is:
N-[3-(1-azido-2-methylpropan-2-yl)-1,2-oxazol-5-yl]-5-cyclopropyl-6-(cyclopropylmethoxy)pyrazine-2-carboxamide;
or a pharmaceutically acceptable salt thereof.

18. A process for the preparation of a compound according to claim 1, comprising reacting a compound of formula (A): 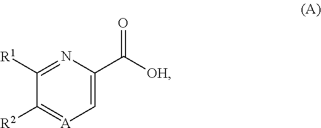
a compound of the formula NHR³R⁴, an amide coupling agent, and a base.

19. A process for the preparation of a compound according to claim 1, wherein R¹ is halophenyl or halophenylalkyl, comprising:
reacting a compound of formula (B): 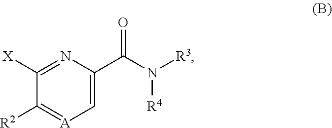
wherein X is Cl, Br, I or trifluoromethanesulfonato;
a compound of the formula R¹—Y, where Y is a trifluoroborate group, a boronic acid group or a boronic acid pinacol ester group; a palladium catalyst; and a base.

20. A process for the preparation of a compound according to claim 1, wherein R¹ is halophenyl, halophenylalkyl or oxopyrrolidinyl; and R² is cycloalkyl, comprising:
reacting a compound of formula (C): 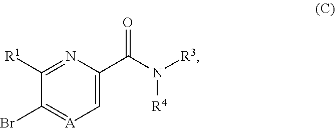
a compound of the formula R²-M, where M is a trifluoroborate group, a boronic acid group or a boronic acid pinacol ester group; a palladium catalyst and a base.
